(19) United States
(12) Patent Application Publication
(10) Pub. No.: US 2021/0322422 A1
(43) Pub. Date: Oct. 21, 2021

(54) USE OF AKT INHIBITORS IN OPHTHALMOLOGY

(71) Applicant: Hoffmann-La Roche Inc., Little Falls, NJ (US)

(72) Inventors: Ashwath JAYAGOPAL, Basel (CH); Debasish SINHA, Clarksville, MD (US)

(73) Assignee: Hoffmann-La Roche Inc., Little Falls, NJ (US)

(21) Appl. No.: 17/229,040

(22) Filed: Apr. 13, 2021

Related U.S. Application Data
(63) Continuation of application No. PCT/EP2019/077683, filed on Oct. 14, 2019.
(60) Provisional application No. 62/746,105, filed on Oct. 16, 2018.

(51) Int. Cl. A61K 31/519; C12N 15/113; A61K 31/7105; A61K 31/713; A61P 27/02

(52) U.S. Cl. CPC A61K 31/519; C12N 15/1137; A61K 31/7105; C12N 2310/531; A61P 27/02; C12N 2310/11; C12N 2310/14; A61K 31/713

(57) ABSTRACT

The present invention provides the use of an Akt inhibitor for the treatment of ocular vascular disease, in particular age-related macular degeneration.

### CROSS-REFERENCE TO RELATED APPLICATIONS

This application is a Continuation of International Application No. PCT/EP2019/077683, filed Oct. 14, 2019, which claims priority to and the benefit of U.S. Application No. 62/746,105, filed Oct. 16, 2018, the entirety of each of which are incorporated herein by reference.

### TECHNICAL FIELD

The present invention relates to the use of Akt inhibitors for the treatment of ocular vascular disease, in particular age-related macular degeneration (AMD).

### BACKGROUND

Age-related macular degeneration (AMD) is a progressive degenerative macular disease attacking the region of highest visual acuity (VA), the macula, and is the leading cause of blindness in Americans 60 years or older (NIH Medline Plus (2008), Leading cause of blindness, NIH Medline Plus 3(2) 14-15. www.nlm.nih.gov/medlineplus/magazine/issues/summer08/articles/summer08pgl4-15.html). The neovascular “wet” form of the disease (nAMD or wet AMD) is characterized by choroidal neovascularization which is marked by proliferation of blood vessels and cells including those of the retinal pigment epithelium (RPE) (Carmeliet (2005) Nature 438: 932-936). Ultimately, photoreceptor death and scar formation result in a severe loss of central vision and the inability to read, write, and recognize faces or drive. Many patients can no longer maintain gainful employment, carry out daily activities and consequently report a diminished quality of life (Mitchell and Bradley (2006), Health Qual Life Outcomes 4: 97). Preventative therapies have demonstrated little effect and therapeutic strategies have focused primarily on treating the neovascular lesion.

Some currently available treatments for wet AMD include laser photocoagulation, photodynamic therapy with verteporfin, and intravitreal (IVT) injections with the vascular endothelial growth factor (VEGF) inhibitors such as pegaptanib, ranibizumab, bevacizumab or aflibercept (Schmidt-Erfurth, (2014) Guidelines for the management of neovascular age-related macular degeneration by the European Society of Retina Specialists (EURETINA) Br J Ophthalmol 98: 1144-1167). While these therapies have some effect on best-corrected visual acuity (BCVA), their effects may be limited in restoring visual acuity and in duration (Schmidt-Erfurth, cited above, 2014, AAO PPP (2015) Preferred Practice Patterns: Age Related Macular Degeneration. American Academy of Ophthalmology).

Several drugs in market that are used to treat wet AMD rely on a mechanism that inhibits VEGF and must be injected intravitreally. While these treatments are reported to succeed in prohibiting the disease from progressing, they require frequent injections of the drug.

### SUMMARY OF THE INVENTION

AKT is a serine-threonine kinase identified as an oncogene in a mouse leukemia virus, and it has been revealed that its activity is important for various functions, such as cell proliferation, survival, metabolism, metastasis, and invasion (Cell, 129, p. 1261-1274 (2007); Cell Cycle. 7. p. 2991-2996 (2008)). In human beings, three isoforms (AKT1/PKBα, AKT2/PKBβ, and AKT3/PKBγ) have been reported (Proc. Natl. Acad. Sci. USA 84. p. 5034-5037 (1987); J. Biol Chem. 274. p. 9133-9136 (1999)). Activation of AKT involves localization to the plasma membrane by binding to PI3 kinase-generated phosphatidylinositol 3-phosphate, and phosphorylation by multiple kinases (FEBS Letters. 546. p. 108-112 (2003)). In many cancers (e.g., breast cancer, pancreatic cancer, liver cancer, prostatic cancer, stomach cancer, lung cancer, ovarian cancer, head and neck cancer, urinary tract cancer, and endometrial cancer), it has been reported that the expression of activated AKT is enhanced by activation of PI3 kinase due to mutation, etc., or inactivation of its negative regulator, PTEN (Nature Reviews Drug Discovery, 8, p. 627-644 (2009)). In addition, enhanced expression of activated AKT has been reported to be associated with poor prognosis in various cancers (e.g., breast cancer, pancreatic cancer, liver cancer, prostatic cancer, stomach cancer, and endometrial cancer) (Anticancer Research, 18, p. 861-874 (2007)).

In the present invention, an Akt inhibitor refers to a molecule capable of inhibiting the expression and/or activity of AKT at nucleic acid level and/or protein level. An Akt inhibitor available in the art can be used in the present invention. For example, suitable small molecule Akt inhibitors are disclosed in EP2698372, US20070185152, US20080255143, US20080269131, US20090227616, US20100056523, US20100137338, US20110053972, US20110071182, WO2005046678, WO2006113837, WO2007076320, WO2007076423, WO2008121685, WO2009032651, WO2009032652, WO2009032653, WO2009158371, WO2009158372, WO2009158373, WO2009158374, WO2009158376, WO2010019637 and George Mihai Nitulescu et al., International Journal of Oncology 48: 869-885, 2016.

Alternatively, an Akt inhibitor may be an mRNA interfering RNA molecule; or may be an antagonist of Akt protein, for example, a ligand, aptamer or antibody. In an embodiment, the Akt inhibitor is an antibody to Akt protein. In another embodiment, the Akt inhibitor is a double-stranded RNA (dsRNA), for example, a short interfering RNA (siRNA) or a short hairpin RNA (shRNA). The double-stranded RNA may be any type of RNA, including but not limited to mRNA, snRNA, microRNA, and tRNA. RNA interference (RNAi) is particularly useful for specifically inhibiting the production of specific RNA and/or proteins. The design and production of dsRNA molecules suitable for the present invention are within the skill of those skilled in the art, particularly with reference to Waterhouse et al. (1998), Smith et al. (2000), WO 99/32619, WO 99/53050, WO 99/49029 and WO 01/34815. Preferably siRNA molecule comprises a nucleotide sequence having about 19 to 23 contiguous nucleotides identical to the target mRNA. The term “shRNA” refers to a siRNA molecule in which fewer than about 50 nucleotides pair with the complementary sequence on the same RNA molecule, which sequence and complementary sequence are separated by an unpaired region of at least about 4 to 15 nucleotides (forming a single-chain loop on the stem structure produced by the two base-complementary regions). There are well-established siRNA design criteria (see, for example, Elbashire et al., 2001).

In a further aspect of the invention, the Akt inhibitor can be an antisense oligonucleotide which is capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides are single stranded. It is understood that single stranded oligonucleotides can form hairpins or intermolecular duplex structures (duplex between two molecules of the same oligonucleotide), as long as the degree of intra or inter self-complementarity is less than 50% across of the full length of the oligonucleotide.

In an embodiment of the present invention, the Akt inhibitor is an Akt2 selective or specific inhibitor. In the present invention, when used for inhibitor, the term “selective” and “specific” can be used interchangeably, meaning that the inhibitor has an inhibitory effect on the target only, or has a higher inhibitory effect on the target than on other compounds or molecules, for example, higher by at least about 2, 3, 4, 5, 6, 7, 8, 9, 10, 20, 30, 40, 50, 100, 500, 1000, 10000 folds, and the like. For example, CCT128930 (Selleckchem) is an effective ATP-competitive selective Akt2 inhibitor, which has an ICSO value of 6 nM in a cell-free assay and exhibits 28 folds of higher selectivity on Akt2 than closely related PKA kinase.

The terms “ocular vascular disease” and “vascular eye disease” are use inter changeable herein and include, but are not limited to intraocular neovascular syndromes such as diabetic retinopathy, diabetic macular edema, retinopathy of prematurity, neovascular glaucoma, retinal vein occlusions, central retinal vein occlusions, macular degeneration, age-related macular degeneration, retinitis pigmentosa, retinal angiomatous proliferation, macular telangectasia, ischemic retinopathy, iris neovascularization, intraocular neovascularization, corneal neovascularization, retinal neovascularization, choroidal neovascularization, and retinal degeneration. (Garner, A., Vascular diseases, In: Pathobiology of ocular disease, A dynamic approach, Garner, A., and Klintworth, G. K., (eds.), 2nd edition, Marcel Dekker, New York (1994), pp. 1625-1710). As used herein, ocular vascular disorder refers to any pathological conditions characterized by altered or unregulated proliferation and invasion of new blood vessels into the structures of ocular tissues such as the retina or cornea. In one embodiment the ocular vascular disease is selected from the group consisting of: wet age-related macular degeneration (wet AMD), dry age-related macular degeneration (dry AMD), diabetic macular edema (DME), cystoid macular edema (CME), non-proliferative diabetic retinopathy (NPDR), proliferative diabetic retinopathy (PDR), cystoid macular edema, vasculitis (e.g. central retinal vein occlusion), papilloedema, retinitis, conjunctivitis, uveitis, choroiditis, multifocal choroiditis, ocular histoplasmosis, blepharitis, dry eye (Sjögren's disease) and other ophthalmic diseases wherein the eye disease or disorder is associated with ocular neovascularization, vascular leakage, and/or retinal edema. So Akt inhibitors according to the invention are useful in the prevention and treatment of wet AMD, dry AMD, CME, DME, NPDR, PDR, blepharitis, dry eye and uveitis, also preferably wet AMD, dry AMD, blepharitis, and dry eye, also preferably CME, DME, NPDR and PDR, also preferably blepharitis, and dry eye, in particular wet AMD and dry AMD, and also particularly wet AMD. In some embodiments, the ocular disease is selected from the group consisting of wet age-related macular degeneration (wet AMD), macular edema, retinal vein occlusions, retinopathy of prematurity, and diabetic retinopathy.

Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, retinitis pigmentosa, retina edema (including macular edema), Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

Retinopathy of prematurity (ROP) is a disease of the eye that affects prematurely born babies. It is thought to be caused by disorganized growth of retinal blood vessels which may result in scarring and retinal detachment. ROP can be mild and may resolve spontaneously, but may lead to blindness in serious cases. As such, all preterm babies are at risk for ROP, and very low birth weight is an additional risk factor. Both oxygen toxicity and relative hypoxia can contribute to the development of ROP.

Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. According to the American Academy of Ophthalmology, it is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD).

Age-related macular degeneration begins with characteristic yellow deposits in the macula (central area of the retina which provides detailed central vision, called fovea) called drusen between the retinal pigment epithelium and the underlying choroid. Most people with these early changes (referred to as age-related maculopathy) have good vision. People with drusen can go on to develop advanced AMD. The risk is considerably higher when the drusen are large and numerous and associated with disturbance in the pigmented cell layer under the macula. Large and soft drusen are related to elevated cholesterol deposits and may respond to cholesterol lowering agents or the Rheo Procedure.

Advanced AMD, which is responsible for profound vision loss, has two forms: dry and wet. Central geographic atrophy, the dry form of advanced AMD, results from atrophy to the retinal pigment epithelial layer below the retina, which causes vision loss through loss of photoreceptors (rods and cones) in the central part of the eye. While no treatment is available for this condition, vitamin supplements with high doses of antioxidants, lutein and zeaxanthin, have been demonstrated by the National Eye Institute and others to slow the progression of dry macular degeneration and in some patients, improve visual acuity.

Retinitis pigmentosa (RP) is a group of genetic eye conditions. In the progression of symptoms for RP, night blindness generally precedes tunnel vision by years or even decades. Many people with RP do not become legally blind until their 40s or 50s and retain some sight all their life. Others go completely blind from RP, in some cases as early as childhood. Progression of RP is different in each case. RP is a type of hereditary retinal dystrophy, a group of inherited disorders in which abnormalities of the photoreceptors (rods and cones) or the retinal pigment epithelium (RPE) of the retina lead to progressive visual loss. Affected individuals first experience defective dark adaptation or nyctalopia (night blindness), followed by reduction of the peripheral visual field (known as tunnel vision) and, sometimes, loss of central vision late in the course of the disease.

Macular edema occurs when fluid and protein deposits collect on or under the macula of the eye, a yellow central area of the retina, causing it to thicken and swell. The swelling may distort a person's central vision, as the macula is near the center of the retina at the back of the eyeball. This area holds tightly packed cones that provide sharp, clear central vision to enable a person to see form, color, and detail that is directly in the line of sight. Cystoid macular edema is a type of macular edema that includes cyst formation.

Combination Therapies: In certain embodiments the Akt inhibitor or pharmaceutical composition according to the invention is administered alone (without an additional therapeutic agent) for the treatment of one or more ocular vascular diseases described herein.

In other embodiments the Akt inhibitor or pharmaceutical composition according to the invention is administered in combination with one or more additional therapeutic agents or methods for the treatment of one or more ocular vascular diseases described herein.

In other embodiments, the Akt inhibitor or pharmaceutical composition according to the invention is formulated in combination with one or more additional therapeutic agents and administered for the treatment of one or more ocular vascular diseases described herein.

In certain embodiments, the combination treatments provided herein include administration the Akt inhibitor or pharmaceutical composition according to the invention is administered sequentially with one or more additional therapeutic agents for the treatment of one or more ocular vascular diseases described herein.

The additional therapeutic agents include, but are not limited to, Tryptophanyl-tRNA synthetase (TrpRS), EyeOOl (Anti-VEGF Pegylated Aptamer), squalamine, RETAANE™ (anecortave acetate for depot suspension; Alcon, Inc.), Combretastatin A4 Prodrug (CA4P), MACUGEN™, MIFEPREX™ (mifepristone-ru486), subtenon triamcinolone acetonide, intravitreal crystalline triamcinolone acetonide, Prinomastat (AG3340-synthetic matrix metalloproteinase inhibitor, Pfizer), fluocinolone acetonide (including fluocinolone intraocular implant, Bausch & Lomb/Control Delivery Systems), VEGFR inhibitors (Sugen), VEGF-Trap (Regeneron/Aventis), VEGF receptor tyrosine kinase inhibitors such as 4-(4-bromo-2-fluoroanilino)-6-methoxy-7-(1-methylpiperidin-4-ylmethoxy)quinazoline (ZD6474), 4-(4-fluoro-2-methylindol-5-yloxy)-6-methoxy-7-(3-pyrrolidin-1 ylpropoxy)quinazoline (AZD2171), vatalanib (PTK787) and SU1 1248 (sunitinib), linomide, and inhibitors of integrin v.beta.3 function and angiostatin.

Other pharmaceutical therapies that can be sued used in combination the Akt inhibitor or pharmaceutical composition according to the invention is administered, include, but are not limited to, VISUDYNE™ with use of a non-thermal laser, PKC 412, Endovion (NeuroSearch A/S), neurotrophic factors, including by way of example Glial Derived Neurotrophic Factor and Ciliary Neurotrophic Factor, diatazem, dorzolamide, Phototrop, 9-cis-retinal, eye medication (including Echo Therapy) including phospholine iodide or echothiophate or carbonic anhydrase inhibitors, AE-941 (AEterna Laboratories, Inc.), Sirna-027 (Sima Therapeutics, Inc.), pegaptanib (NeXstar Pharmaceuticals/Gilead Sciences), neurotrophins (including, by way of example only, NT-4/5, Genentech), Cand5 (Acuity Pharmaceuticals), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celltech Group pic), ATX-S10 (Hamamatsu Photonics), TGF-beta 2 (Genzyme/Celtrix), tyrosine kinase inhibitors (Allergan, SUGEN, Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), cyclosporin A, Timited retinal translocation”, photodynamic therapy, (including, by way of example only, receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; motexafin lutetium, Pharmacyclics, Inc.), antisense oligonucleotides (including, by way of example, products tested by Novagali Pharma SA and ISIS-13650, Isis Pharmaceuticals), laser photocoagulation, drusen lasering, macular hole surgery, macular translocation surgery, implantable miniature telescopes, Phi-Motion Angiography (also known as Micro-Laser Therapy and Feeder Vessel Treatment), Proton Beam Therapy, microstimulation therapy, Retinal Detachment and Vitreous Surgery, Scleral Buckle, Submacular Surgery, Transpupillary Thermotherapy, Photosystem I therapy, use of RNA interference (RNAi), extracorporeal rheopheresis (also known as membrane differential filtration and Rheotherapy), microchip implantation, stem cell therapy, gene replacement therapy, ribozyme gene therapy (including gene therapy for hypoxia response element, Oxford Biomedica; Lentipak, Genetix; PDEF gene therapy, GenVec), photoreceptor/retinal cells transplantation (including transplantable retinal epithelial cells, Diacrin, Inc.; retinal cell transplant, Cell Genesys, Inc.), and acupuncture.

Any anti-angiogenic agent can be used in combination with the Akt inhibitor or pharmaceutical composition according to the invention, including, bu not limited to, those listed by Carmeliet and Jain, 2000, Nature 407:249-257. In certain embodiments, the anti-angiogenic agent is an VEGF antagonist or a VEGF receptor antagonist such as VEGF variants, soluble VEGF receptor fragments, aptamers capable of blocking VEGF or VEGFR, neutralizing anti-VEGFR antibodies, low molecule weight inhibitors of VEGFR tyrosine kinases and any combinations thereof and these include anti-VEGF aptamers (e.g. Pegaptanib), soluble recombinant decoy receptors (e.g. VEGF Trap). In certain embodiments, the anti-angiogenic agent is include corticosteroids, angiostatic steroids, anecortave acetate, angiostatin, endostatin, small interfering RNA's decreasing expression of VEGFR or VEGF ligand, post-VEGFR blockade with tyrosine kinase inhibitors, MMP inhibitors, IGFBP3, SDF-1 blockers, PEDF, gamma-secretase, Delta-like ligand 4, integrin antagonists, HIF-1 alpha blockade, protein kinase CK2 blockade, and inhibition of stem cell (i.e. endothelial progenitor cell) homing to the site of neovascularization using vascular endothelial cadherin (CD-144) and stromal derived factor (SDF)-I antibodies. Small molecule RTK inhibitors targeting VEGF receptors including PTK787 can also be used. Agents that have activity against neovascularization that are not necessarily anti-VEGF compounds can also be used and include anti-inflammatory drugs, m-Tor inhibitors, rapamycin, everolismus, temsirolismus, cyclospohne, anti-TNF agents, anti-complement agents, and nonsteroidal antiinflammatory agents. Agents that are neuroprotective and can potentially reduce the progression of dry macular degeneration can also be used, such as the class of drugs called the ‘neurosteroids.’ These include drugs such as dehydroepiandrosterone (DHEA)(Brand names: Prastera® and Fidelin®), dehydroepiandrosterone sulfate, and pregnenolone sulfate. Any AMD (age-related macular degeneration) therapeutic agent can be used in combination with the Akt inhibitor or pharmaceutical composition according to the invention, including but not limited to verteporfin in combination with PDT, pegaptanib sodium, zinc, or an antioxidant(s), alone or in any combination.

The terms “subject” and “patient” are used interchangeably and refer to mammals such as human patients and non-human primates, as well as experimental animals such as rabbits, rats, and mice, and other animals. Animals include all vertebrates, e.g., mammals and nonmammals, such as dogs, cats, sheeps, cows, pigs, rabbits, chickens, and etc. Preferred subjects for practicing the therapeutic methods of the present invention are human. Subjects in need of treatment include patients already suffering from an ocular vascular disease or disorder as well as those prone to developing the disorder.

The Akt inhibitor and the pharmaceutically acceptable salts of the Akt inhibitor can be used as medicaments, e.g. in the form of pharmaceutical preparations. The pharmaceutical preparations can be administered orally, e.g. in the form of tablets, coated tablets, dragées, hard and soft gelatine capsules, solutions, emulsions or suspensions. The administration can, however, also be effected rectally, e.g. in the form of suppositories, parenterally, e.g. in the form of injection solutions. The administration can also be effected topically, e.g. transdermal administration, or in form of eye drops or ear drops.

The Akt inhibitor can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing an Akt inhibitor or a pharmaceutically acceptable salt thereof and a therapeutically inert carrier are also an object of the present invention, as is a process for their production, which comprises bringing one or more Akt inhibitor and/or pharmaceutically acceptable acid addition salts and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration, the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

Tablet Formulation (Wet Granulation)

| Item | Ingredients | mg/tablet | | | |
|---|---|---|---|---|---|
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

Manufacturing Procedure
1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

Capsule Formulation

| Item | Ingredients | mg/capsule | | | |
|---|---|---|---|---|---|
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

Manufacturing Procedure
1. Mix items 1, 2 and 3 in a suitable mixer for 30 minutes.
2. Add items 4 and 5 and mix for 3 minutes.
3. Fill into a suitable capsule.

The invention further provides kits including an Akt inhibitor and instructions (e.g., on a label or package insert such as instructions to the subject or to the clinician) for administering the Akt inhibitor to a subject in order to treat, prevent, and/or delay the development or progression of AMD.

An effective amount is a dosage of the Akt inhibitor sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health practitioner.

### BRIEF DESCRIPTION OF THE DRAWINGS

FIGS. 1A-1D depict neutrophil infiltration into the retina of a mouse model with an aging-related non-neovascular AMD-like phenotype and in human early AMD donor tissues. FIG. 1A: Flow cytometry analysis revealed increased percentage of neutrophils (Ly6G+; V450 and CD11b (Alexa fluor 700), Ly6G double positive cells) among the leukocyte (CD45+; FITC cells) gated population in the aged (15 months old) Cryba1 cKO mouse retina compared to aged-matched controls (Cryba1^{fl/fl}). No such changes were observed between Cryba1^{fl/fl} and cKO retinas at 3 months of age. Mean±S.D. for n=4 biological replicates. FIG. 1B(i-iii): Immunofluorescent labeling of tissue sections from early AMD donors with antibodies to NE (neutrophil specific marker-Alexa fluor 488; Green) and MPO (activated neutrophil marker-Alexa fluor 555; Red) showed marked association of neutrophils (FIG. 1B (i)) with endothelial cells (white arrows) of retinal blood vessels (asterisk), and (FIG. 1B (ii)) on the surface of drusen deposits under the retina (white arrows). However, in control samples (FIG. 1B (iii)), fewer neutrophils were detected (asterisk), and they were not positive for MPO. n=3; Scale bars: 50 μm. FIG. 1C: ELISA revealed increased levels (pg ml⁻¹) of; (i) CXCL1, (ii) IFNα and (iii) IFNλ, in the RPE-choroid tissue homogenate of 15-month-old Cryba1 cKO mice compared to age-matched Cryba1^{fl/fl} controls. No statistically significant changes were observed in 3-month-old mice. Mean±S.D, n=4. FIG. 1D: Representative immunoblot and bar graph showing elevated expression of CXCL1 and IFNλ, in RPE lysates from early AMD donor samples compared to age-matched controls. Mean±S.D; n=3. *P<0.05; **P<0.01. All P-values were evaluated by one-way ANOVA and Tukey's post-hoc test. Ly6G: Lymphocyte antigen 6 complex locus G6D; CD11b: cluster of differentiation 11b; CD45: cluster of differentiation 45; fl/fl: floxed/floxed; cKO: conditional knockout; NE: neutrophil elastase; MPO: myeloperoxidase; DAPI: 4′,6-diamidino-2-phenylindole; AMD: age-related macular degeneration; CXCL1: chemokine (C-X-C motif) ligand 1, IFNα: Interferon alpha; IFNλ: Interferon lambda.

FIGS. 2A-2H depict increased levels of Interferons (IFNs) and activated IL-28R1+ neutrophils in AMD patients. Levels of IFNα (FIG. 2A), IFNβ (FIG. 2B), IFNγ (FIG. 2C), IFNλ1 (FIG. 2D) and IFNλ2/3 (FIG. 2E) in plasma from early AMD subjects (without geographic atrophy or neovascularization; n=50) and controls (without AMD or diabetes; n=26) measured by multiplex ELISA. Levels of IFNα (FIG. 2F), and IFNλ1 (FIG. 2G) in aqueous humor (Aq. H) from early AMD subjects (cataract subjects with small drusen and pigmentary changes in retina; n=6) compared to control (cataract subjects without any retinal pathology; n=7). Immune cell populations in peripheral blood (PB) and Aq. H (50 μL) were stained for antibodies to CD45 (leukocytes), CD66b (neutrophils) and IL-28R1 (IFNλ, receptor 1) and gated for the following sub-populations: CD45⁺ CD66b⁺ (FIG. 211) in PB; CD45⁺ IL-28R1⁺(i) in PB; CD45⁺ CD66b^{High} IL-28R1⁺(j) in PB and CD45⁺ CD66b^{High} IL-28R1⁺ (k) in Aq. H. *P<0.05, **P<0.01, ***P<0.001, ****P<0.0001, Mann Whitney Test. ELISA: Enzyme-linked immunosorbent Assay; CD: cluster of differentiation; IL-28R1: Interleukin 28 receptor 1.

FIGS. 3A-3E depicts activation of LCN-2/Dab2/integrin β1 axis elicits transmigration of neutrophils into the retina leading to retinal degeneration. FIG. 3A: Neutrophils exposed (6 h) to conditioned media from IFNλ, overexpressing RPE cells or to recombinant IFNλ, (2 h), showed increased expression of LCN-2 and pSTAT1 with respect to control cells. Mean±S.D, n=3; FIG. 3B: Representative fundus and spectral-OCT images of retinas from NOD-SCID mice that were administered intravitreal injections with (i) vehicle (HBSS) or (ii) control neutrophils revealed normal retinal structure. In contrast, mice injected with; (iii) recombinant LCN-2 (10 pg ml⁻¹) or neutrophils pre-treated with either (iv) conditioned media (diluted 1:1) from IFNλ, overexpressing mouse RPE cells or (v) recombinant IFNλ, (200 U ml⁻¹), show apparent merging of RPE with IS/OS layer (asterisks), large focal nodules in the GCL/IPL layer (yellow arrows) and focal protrusions in the IS/OS/RPE layers extending into the ONL (white arrows). FIG. 3C: Pull down assay of cell lysate from mouse bone-marrow derived cultured neutrophils, treated as in FIG. 3B, showed increased association between LCN-2 (immunoprecipitated) and Dab2 (immunoblotted). Negative control: Rabbit IgG; input controls for each sample show presence of Dab2 in the un-immunoprecipitated neutrophil lysates. Mean±S. D.; n=3. FIG. 3D: Flow cytometry analysis of mouse neutrophils exposed to conditioned media as above showed increased extracellular expression of integrin β1 (FITC-A Median fluorescence); however, 8 h following LCN-2 shRNA transfection integrin β1 was reduced, compared to control shRNA transfected neutrophils. Mean±S. D.; n=3. FIG. 3E: As in FIG. 3D, neutrophils treated with recombinant IFNλ, show marked increase in integrin (31, which was largely prevented by transfection with LCN-2 shRNA. Mean±S. D.; n=3. *P<0.05, P-values were evaluated by one-way ANOVA and Tukey's post-hoc test. LCN-2: Lipocalin-2; shRNA: small/short hairpin RNA; NOD-SCID: NOD-severe combined immunodeficiency; IFNλ: Interferon lambda, IS: photoreceptor inner segment; OS: photoreceptor outer segment; GCL: ganglion cell layer; IPL: inner plexiform layer; OLM: outer limiting membrane; ONL: outer nuclear layer; Dab-2: disabled homolog 2; V450: violet 450; FITC: fluorescein isothiocyanate.

FIGS. 4A-4D depict a selective inhibitor of AKT2 phosphorylation blocks neutrophil infiltration into the retina, neutralizes inflammatory signals and rescues early RPE changes in Cryba1 cKO mice. FIG. 4A: Flow cytometry analysis showed decrease in infiltrating neutrophils (Ly6G+; V450 and CD11b (Alexa fluor 700), Ly6G double positive cells) within the leukocyte (CD45+; FITC cells) gated population, in retinas of Cryba1 cKO mice (Male; 12 months old) after intravitreal treatment with AKT2 inhibitor (CCT128930) at a dose of 500 uM/2 uM, once weekly for three weeks, compared to vehicle treated (PBS containing 2.5% DMSO in PBS) or untreated age-matched Cryba1 cKO. Representative graphs denote % Ly6G+ and % CD11b+Ly6G+ cells (Mean±S.D.) for n=3 biological replicates. FIG. 4B: Representative histological sections (H&E) of retina from 1 year old Cryba1^{fl/fl} mouse showing normal structure FIG. 4C: Cryba1 cKO mouse (1 year old) injected with vehicle (as above) shows photoreceptor and RPE lesions with pigmentation changes (arrows). Inset shows higher magnification of RPE lesions indicating possible debris accumulation between Bruch's membrane and RPE as well as separation of photoreceptors from RPE (arrows) FIG. 4D: In contrast, Cryba1 cKO mice injected with CCT128930, exhibited normal structure after 2 weeks. Original magnification: (FIGS. 4B-4D 20×; (Inset) 40×. *P<0.05, all P-values were evaluated by one-way ANOVA and Tukey's post-hoc test. Ly6G: Lymphocyte antigen 6 complex locus G6D; CD: cluster of differentiation 11b; fl/fl: floxed/floxed; cKO: conditional knockout; AKT2: AKT Serine/Threonine Kinase 2; GCL: ganglion cell layer; IPL: inner plexiform layer; INL: inner nuclear layer; OPL: outer plexiform layer; ONL: outer nuclear layer; IS/OS: inner and outer segment of photoreceptor; RPE/BrM/CC: retinal pigment epithelium, Bruch's membrane, choriocapillaris complex.

FIG. 5 depicts increased expression of neutrophil adhesion molecule in retinas of mice with AMD-like pathology. Immunofluorescence of ICAM-1 (Alexa fluor 555), a cellular adhesion molecule required for neutrophil homing into inflamed tissue, shows elevated expression in the retina of aged (18 months old) Cryba1 cKO mice), but not in 7 month old mice (top panel), as compared to age matched Cryba1fl/fl (control) mice. Intense ICAM-1 staining was noticed in the neural retina (arrows) and in the RPE/choroid (asterisk) of the aged Cryba1 cKO mice (bottom right). The images are representative of three biological replicates. Scale bars: 50 μm. ICAM-1: intercellular adhesion molecule 1; DAPI: 4′,6-diamidino-2-phenylindole; GCL: ganglion cell layer; INL: inner nuclear layer; ONL: outer nuclear layer; RPE: retinal pigment epithelium.

FIGS. 6A-6B depict increased expression of neutrophil extracellular traps (NETs) in retina of human AMD patients. FIG. 6A: In sections from human donor eyes (see Methods for details), immunofluorescence demonstrates that AMD retinas have increased number of cells positive for neutrophil elastase (NE: Alexa fluor 555), a neutrophil specific marker and for H3 citrunilated histone (Alexa fluor 488; Green), a component of NETs, as compared to age-matched controls. FIG. 6B: Sections from human AMD retina also revealed increased MPO (Alexa fluor 555) and H3 citrunilated histone (Alexa fluor 488) double positive cells (arrows) which are markers for activated neutrophils and are components of NETs. No such staining was observed in control samples (data not shown). n=3; scale bars: 50 μm. AMD: age-related macular degeneration; H3: histone 3; MPO: myeloperoxidase; DAPI: 4′,6-diamidino-2-phenylindole; GCL: ganglion cell layer; INL: inner nuclear layer; ONL: outer nuclear layer; RPE: retinal pigment epithelium.

FIG. 7 depicts increased RNA expression of neutrophil-regulating molecules in the RPE/Choroid of mice with AMD-like pathology. RNASeq analysis revealed significant increase in RNA levels of neutrophil regulating molecules like CXCL1, CXCL9 and IFNγ, IFNα and IFNλ, in RPE/choroid extracts from 10-month-old Cryba1 cKO mice compared to age-matched Cryba1^{fl/fl} (control). No such changes were observed in 5 month old mice. All values represent Reads Per Kilobase of transcript per Million mapped reads (RPKM) for each gene and are represented as log₁₀ (counts per million); n=6. **P<0.01 and ns: not significant with respect to 10-month-old Cryba1^{fl/fl} group. All P-values were evaluated by one-way ANOVA and Tukey's post-hoc test. CXCL1: chemokine (C-X-C motif) ligand 1; CXCL9: chemokine (C-X-C motif) ligand 9; IFNγ: interferon gamma; IFNα: interferon alpha; IFNλ: interferon lambda.

FIGS. 8A-8D depict IFNβ, IFNλ2/3 and VEGF levels in aqueous humor and plasma of AMD patients. Levels of IFNβ (FIG. 8A) and IFNλ, 2/3 (FIG. 8B) in aqueous humor of early AMD subjects (n=6) and controls (n=7) measured by multiplex ELISA showed no significant changes. Similarly, levels of VEGF in peripheral blood (FIG. 8C) and aqueous humor (FIG. 8D) of early AMD subjects and controls (see methods for details) measured by multiplex ELISA showed no differences. INFβ: interferon beta; IFNλ: interferon lambda; VEGF: vascular endothelial growth factor.

FIGS. 9A-9F depict immune cell analysis in aqueous humor and plasma. Immune cell populations in the aqueous humor from AMD subjects (n=6) and controls (n=7) were stained for CD45 (leukocytes) and CD66b (neutrophils) and gated for the following sub-populations: CD45⁺ CD66b⁺ (FIG. 9A-aqueous humor), CD45⁺ CD66b^{High} (FIG. 9B-aqueous humor), CD45⁺ (FIG. 9C-aqueous humor and FIG. 9D-plasma) and CD45⁺ IL28R1⁺ (FIG. 9E—plasma and FIG. 9F—aqueous humor).

FIGS. 10A-10B depicts IS/OS and GCL/IPL thickness measurements of untreated and neutrophil-treated NOD-SCID mice. Thickness analysis was performed on optical sections (100 sections per retina) from each eye ranging from −2.0 to +2.0 mm with respect to the optic nerve head (ONH) using the FIJI-ImageJ (NIH) plugin provided with the instrument along with Diver 2.4 software (Bioptigen). Intravitreal injections with Recombinant LCN-2, neutrophils exposed to conditioned media or neutrophils exposed to recombinant IFNλ, caused; FIG. 10A: decrease in IS+OS thickness (μm) and FIG. 10B: increase in GCL+IPL thickness (μm) as compared to vehicle and untreated (control) neutrophil injected groups. The values are Mean±S.D; n=10. *P<0.05, **P<0.01. All P-values were evaluated by one-way ANOVA and Tukey's post-hoc test. LCN-2: lipocalin-2; IFNλ: interferon lambda; IS: inner segment; OS: outer segment; GCL: ganglion cell layer; IPL: inner plexiform layer.

FIGS. 11A-11C depict histopathologic changes in NOD-SCID mouse eye injected with neutrophils overexpressing recombinant IFNλ. Representative hematoxylin and eosin-stained sections are shown from (FIG. 11A) Wild-type retinal tissue showing normal structure at post-natal day 45. In contrast, (FIG. 11B) NOD-SCID mouse retina injected with neutrophils overexpressing recombinant IFNλ, show infiltrating cells in ganglion cell layer and vitreous (arrowhead), INL, and diffuse photoreceptor damage (arrows) and in (FIG. 11C) severe outer retinal and RPE/BrM complex damage (disruption of BrM, RPE loss, CC changes). FIGS. 11A-B: X10 magnification, FIG. 11c: X20. BrM: Bruch's Membrane; CC: Choriocapilaris; GCL: Ganglion cell layer; IFNλ: interferon lambda; INL: inner nuclear layer; RPE: retinal pigment epithelium.

FIG. 12 depicts a human proteome array showing that Dab-2 interacts with LCN-2. Human proteome array showing binding partners of LCN-2 including Dab-2 (box) probed on HuProt™ arrays at 1 μg/ml. The data is representative of three biological replicates and is represented as z-score (hit for each probe), with a cut-off of 6 and values ranging from 28 to 65.

FIGS. 13A-13B shows that IFNλ, promotes increased adhesion and transmigration in mouse bone marrow-derived neutrophils. Mouse bone-marrow derived neutrophils transfected with control shRNA or LCN-2 shRNA for 8 h and then exposed to conditioned media (diluted 1:1) from control vector or IFNλ, overexpressing RPE cells for 6 h and recombinant IFNλ, (200 U ml⁻¹) for 2 h respectively, showed; FIG. 13A: increased adhesion (arrow) to fibrinogen (20 μg ml⁻¹) coated plates, graph denoting adherent cells, counted in 0.2 mm², using computer-assisted enumeration and FIG. 13B: increase in neutrophil transmigration (arrow) across fibrinogen (150 μg ml⁻¹) coated plates in the control shRNA transfected+IFNλ, (conditioned media or recombinant) neutrophils, graph denoting relative migration (%) of cells representative of cell count at the bottom of insert using a computer assisted cell counter system. LCN-2 shRNA transfected cells showed reduced; FIG. 13A: adhesion (asterisk) and FIG. 13B: transmigration (asterisk) even after exposure to IFNλ. All values are Mean±S.D, n=4; scale bar: 30 μm. *P<0.05, **P<0.01. All P-values were evaluated by one-way ANOVA and Tukey's post-hoc test. LCN-2: lipocalin-2; shRNA: small/short hairpin RNA; IFNλ: interferon lambda; RPE: retinal pigment epithelium; fMLP: N-Formylmethionyl-leucyl-phenylalanine.

FIGS. 14A-14C depict pAKT2, IFNλ, and CXCL1 levels were reduced by CCT128930 treatment. FIG. 14A: Immunoblot and summary of densitometry showing a significant increase in the phosphorylation of AKT2 (p-AKT2^{S474}) in retinas from 1-year-old Cryba1 cKO mice. Treatment with CCT128930 significantly decreased the levels of pAKT2 in the Cryba1 cKO retinas, but not in vehicle controls. Additionally, levels of total AKT did not change in the samples. FIG. 14B & FIG. 14C: ELISA assays show reduced levels (pg ml¹) of CXCL1 (c) and IFNλ, (d) in the RPE-choroid of AKT2 inhibitor treated Cryba1 cKO mice, as compared to age-matched vehicle and untreated Cryba1 cKO animals. Graphs denote values as Mean±S.D. for n=3 biological replicates.

### DETAILED DESCRIPTION OF THE INVENTION

Vision loss from age-related macular degeneration (AMD) is an expanding, major unmet problem due to the aging population¹⁻³. The role of inflammation has emerged as a potential cause of AMD, but how inflammation causes vision loss in AMD remains elusive⁴⁻⁷. In a high-throughput array, we identified inflammatory signals that drive neutrophil infiltration into the retina in early AMD patients and in a mouse model with an aging-related non-neovascular AMD-like phenotype. We observed increased levels of IFNλ, and activated IL28R1+ neutrophils in early AMD. IFNλ, triggers the activation of lipocalin-2 (LCN-2) in neutrophils. LCN-2 potentiates the transmigration of the neutrophils into the retina by interacting with Disabled homolog-2 (Dab2) and modulates integrin b1, promoting the chronic inflammation-induced pathology leading to retinal degeneration. Inhibiting AKT2-dependent signaling in the mouse model neutralizes inflammatory signals, halts neutrophil infiltration into the retina, and reverses early AMD-like phenotype changes, thereby providing a potential therapeutic target for early AMD.

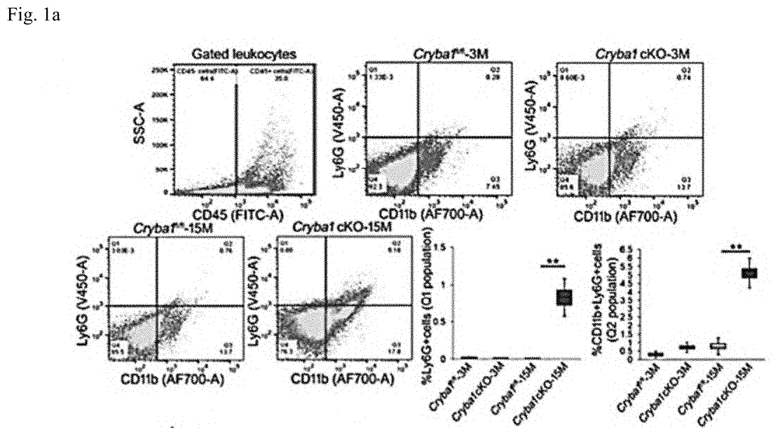

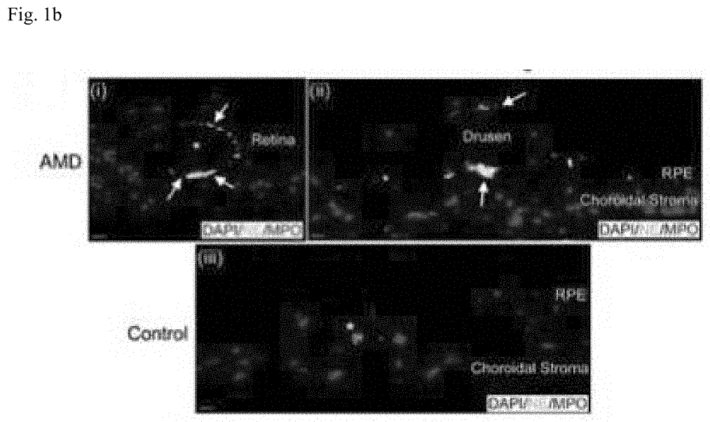

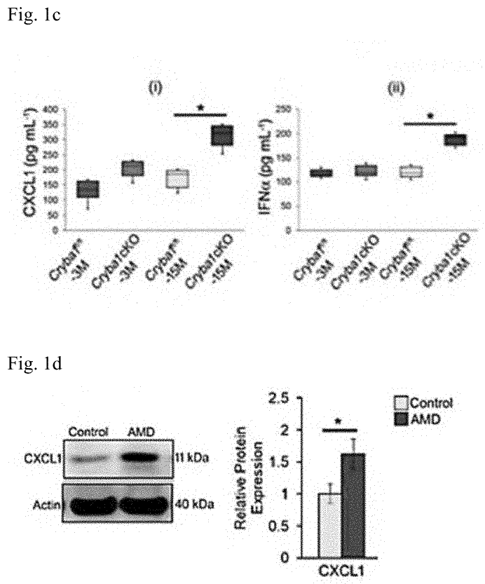

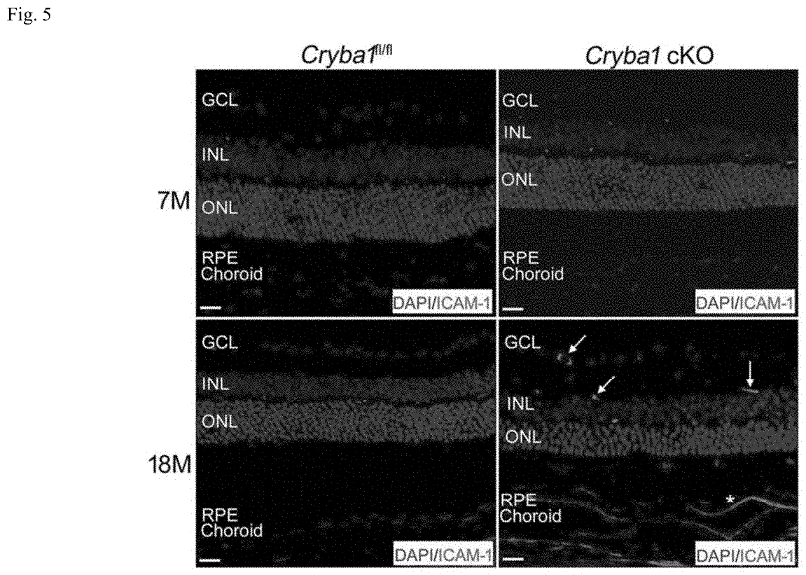

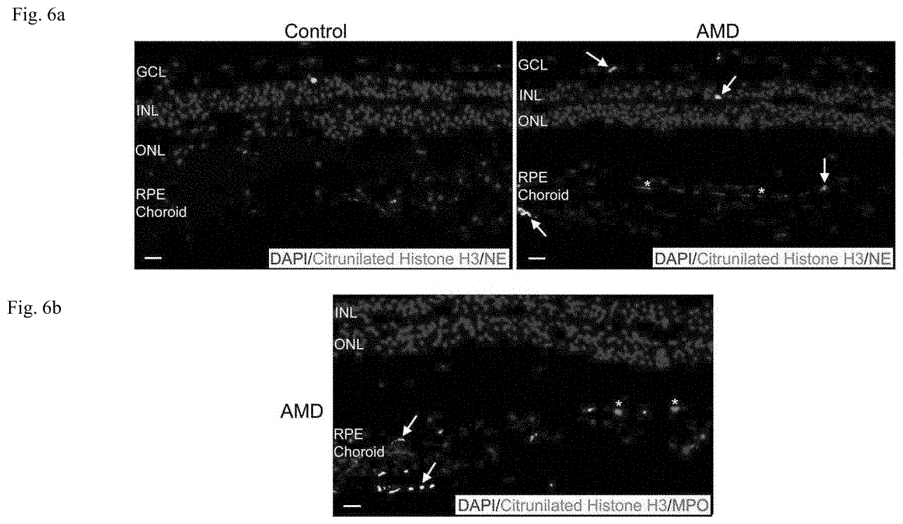

Neutrophils play a central role in the innate immune response⁸⁻¹². It is now clear that dysfunctional circulating neutrophils contribute to pathogenesis in Alzheimer's disease (AD)13-16 and AMD¹⁷⁻¹⁸. In our mouse model (Cryba1 cKO), with an age-dependent AMD-like phenotype¹⁹⁻²¹, flow cytometry demonstrated that the retina accumulates neutrophils, compared to floxed controls (FIG. 1A). We previously observed increased numbers of neutrophils that infiltrate the sub-macular choroid and retina of early AMD patients as compared to age-matched controls²². Herein, we find in retinal tissue sections from patients with non-neovascular AMD, larger numbers of neutrophils associated with the lumen of retinal blood vessels (FIG. 1B) and surrounding drusen, a hallmark AMD lesion (FIG. 1B), relative to age-matched controls (FIG. 1B). Neutrophils transmigrate from the peripheral blood through the endothelial layer towards the site of inflammation by adhering to endothelial cell surfaces and migrate until they crawl along pericytes which signal them to exit through the vessel wall²³⁻²⁷. Neutrophil adhesion to endothelial cells is mediated by interactions with their integrins and immunoglobulin superfamily members28, such as ICAM-129, on endothelial cells. Adhesion molecules are upregulated in our animal model (ICAM-1; FIG. 5). Once in the inflammatory zone, neutrophils can release Neutrophil Extracellular Traps (NETs)³⁰, which recently have been shown to damage host tissue in immune-mediated diseases^{31, 32}. In chronic inflammatory disorders, enhanced NET formation and/or degradation are known to play key roles in the initiation of organ damage33-36. Indeed, AMD donor eyes stained positively for NETs including myeloperoxidase (MPO), elastase and citrullinated histone H3 (FIG. 1B i & ii and FIGS. 6A-6B³⁷⁻⁴⁰.

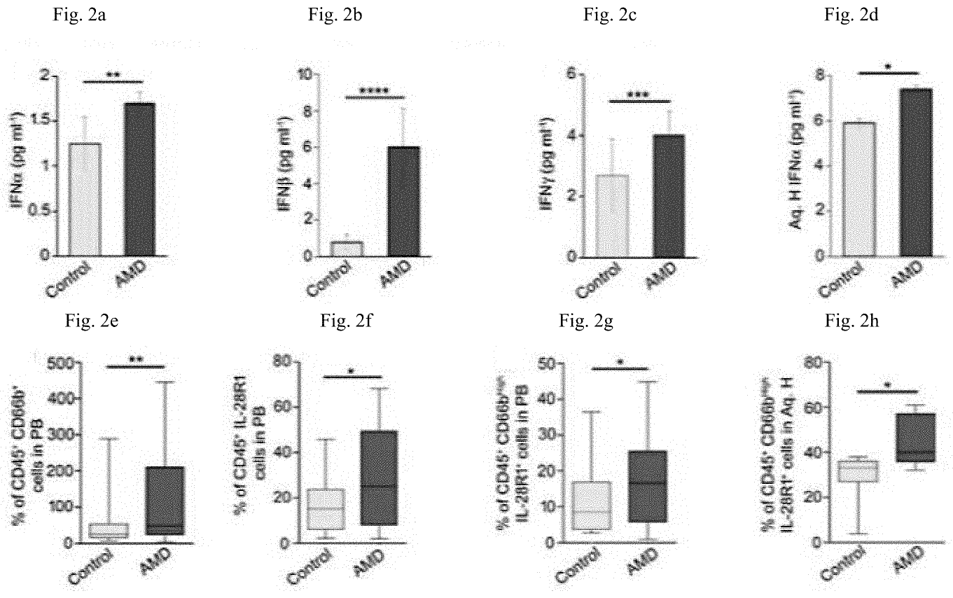

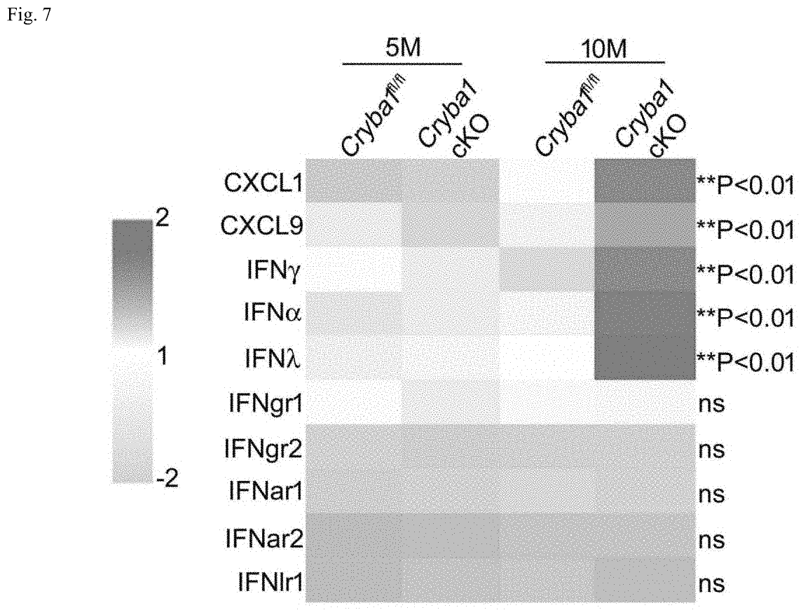

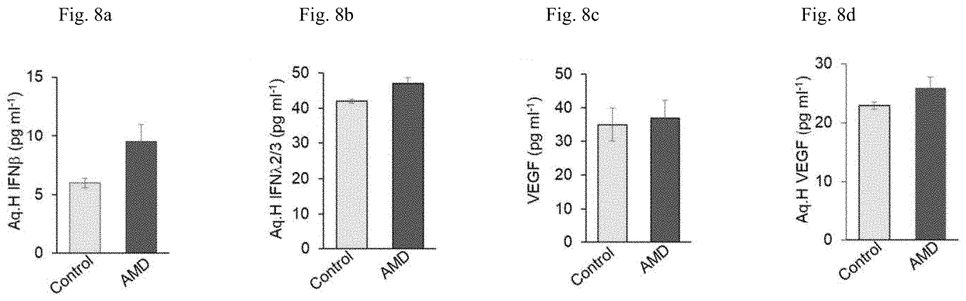

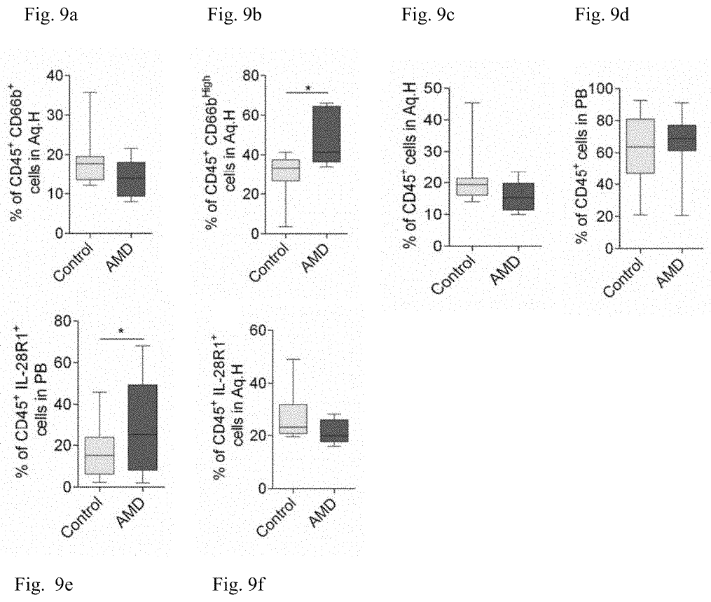

To identify soluble factor(s), such as cytokine(s) or chemokine(s) released from the RPE/retina that may cause neutrophil infiltration into the neurosensory retina and RPE/choroid, we performed RNAseq analysis on retinal tissue obtained from Cryba1 cKO mice¹⁹⁻²¹ and floxed controls4l at 5 and 10 months. The expression of interferons (IFN) as well as CXCL1 and CXCL9 were also increased in the cKO retinas (FIG. 7). These findings were confirmed by ELISA (FIG. 1C). INF1 and CXCL1 were also upregulated in human AMD retinas compared to age-matched controls by western analysis (FIG. 1D). Furthermore, IFNα, IFNβ, IFNγ, IFNλ1 and IFNλ2/3 were increased in the peripheral blood from AMD patients (FIGS. 2A-2E; Supplementary Table 1a) without geographic atrophy or neovascularization (n=50) compared to controls (n=26). In addition, a small set of aqueous humor samples from early AMD subjects (n=6; Supplementary Table 1b) showed elevated IFNα and IFNλ1 compared to age-matched controls (n=7) (FIGS. 2F-2G). IFNβ and IFNλ2/3 were mildly elevated compared to controls, but were not statistically significant, likely due to the small sample size (FIGS. 8A-8B). Interestingly, VEGF levels in patients and controls were not different (FIGS. 8C-8D). Total neutrophils (CD45+/CD66b+) and activated neutrophils (CD66bHigh) were significantly higher in peripheral blood of AMD patients (FIG. 211), but not in aqueous humor (FIGS. 9A-9B). However, the number of IFNλ. receptor-positive (IL-28R1) activated neutrophils was significantly higher within the neutrophil population in both peripheral blood and aqueous humor from AMD subjects compared to controls. Furthermore, the total number of IL28R1-positive cells was not altered in aqueous humor, but elevated in the peripheral blood (FIGS. 9E-9F) Notably, total leukocyte numbers were also not altered in either plasma or aqueous humor in AMD patients compared to controls (FIGS. 9C-9D). Taken together, it is highly likely that IFNλ, is the trigger that potentiates the transmigration of the neutrophils into the retina and possibly the ocular chamber.

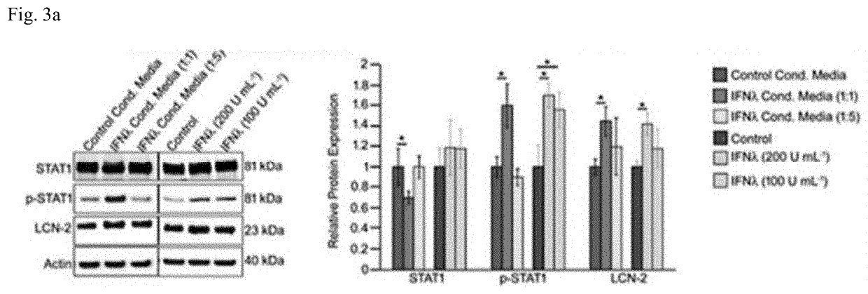

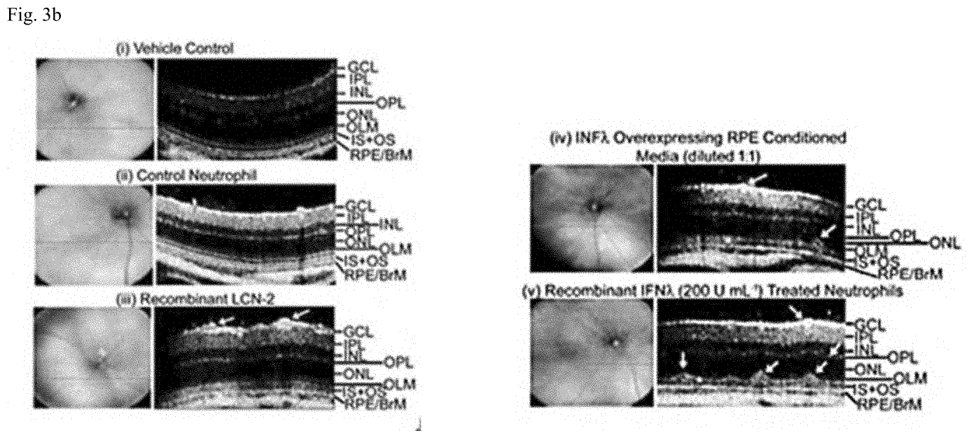

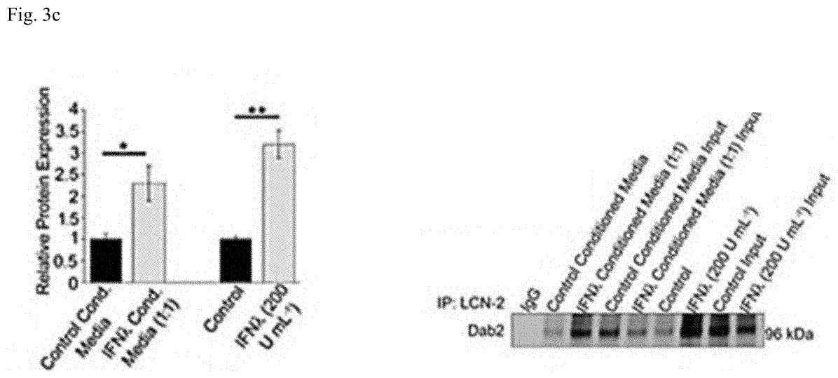

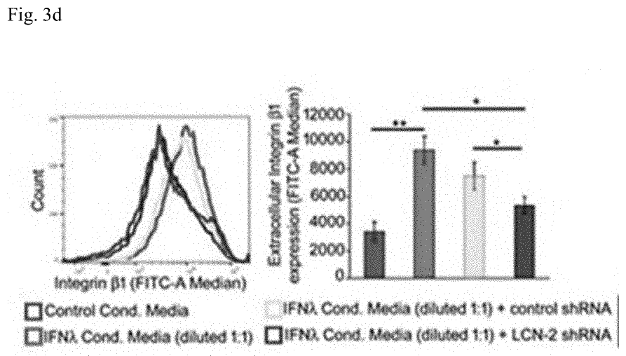

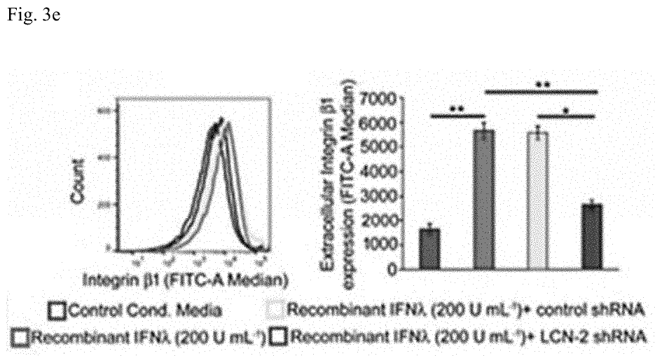

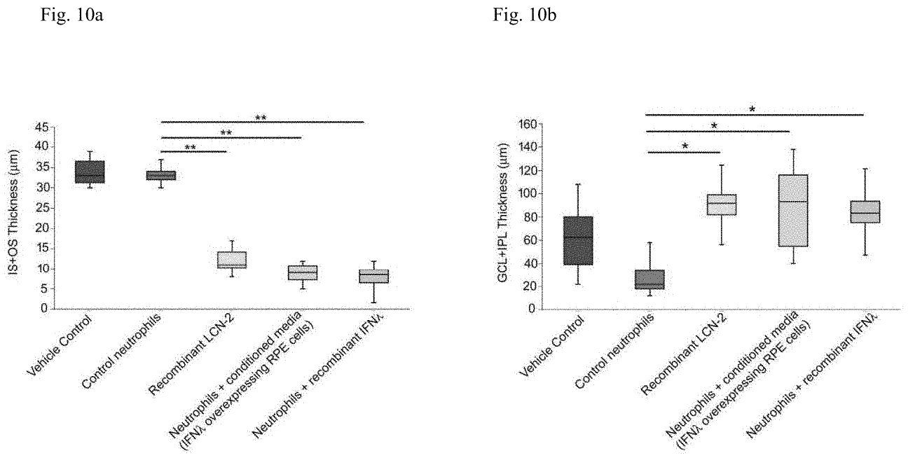

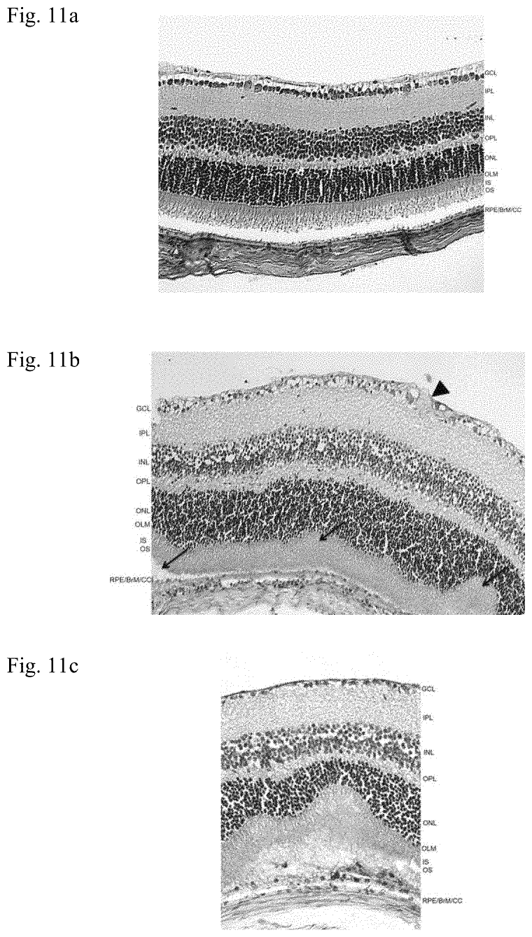

These observations prompted us to investigate possible mechanisms by which neutrophils infiltrate the retina and contribute to the pathogenesis of AMD. In a recent study, we showed that LCN-2 contributes to chronic retinal inflammation in Cryba1 cKO mice42 and that the infiltrating neutrophils in the neurosensory retina and sub-macular choroid of early AMD donor eyes immunostained for LCN-222. Moreover, mouse bone marrow derived neutrophils treated with either recombinant IFNλ, or with conditioned medium from primary cultured RPE cells that overexpress IFNλ, had increased LCN-2 and phosphorylated STAT1 (FIG. 3A). To substantiate our hypothesis that LCN-2 in the transmigrating neutrophils causes outer retinal degeneration, we injected NOD-SCID immunodeficient mice with normal neutrophils, recombinant LCN-2, or neutrophils either treated with INF1 or with conditioned medium from primary cultures of RPE cells overexpressing INFl. After 7 days, the outer retina of mice exposed to neutrophils treated with either INF1 (FIG. 3B iv-v and FIGS. 10A-10B) or recombinant LCN-2 (FIG. 3B iii) had nodular thickening, while no changes were observed in mice treated with normal neutrophils or vehicle only (FIG. 3B i&ii) using Optical Coherence Tomography (OCT). In these outer retinal nodules, the RPE appears to merge with the inner segment (IS)/outer segment (OS) layer, which protruded focally into the outer nuclear layer (FIG. 3B v; white arrows). Focal nodules were also present in the ganglion cell/inner plexiform layers (GCL/IPL). These changes correlated with severe disruption of the outer retina including photoreceptors, the RPE, and Bruch's membrane, and infiltrating cells in the GCL and vitreous (FIGS. 11A-1C). These in vivo findings are reminiscent of the retinal changes observed in the human disease and confirm the pathological role of activated neutrophils in the intraocular compartment and retina of AMD patients. It is tempting to speculate based on the data from NOD-SCID mice and aqueous humor samples from early AMD that activated neutrophils can also migrate into the retina via the intraocular lymphatic drainage system during early AMD.

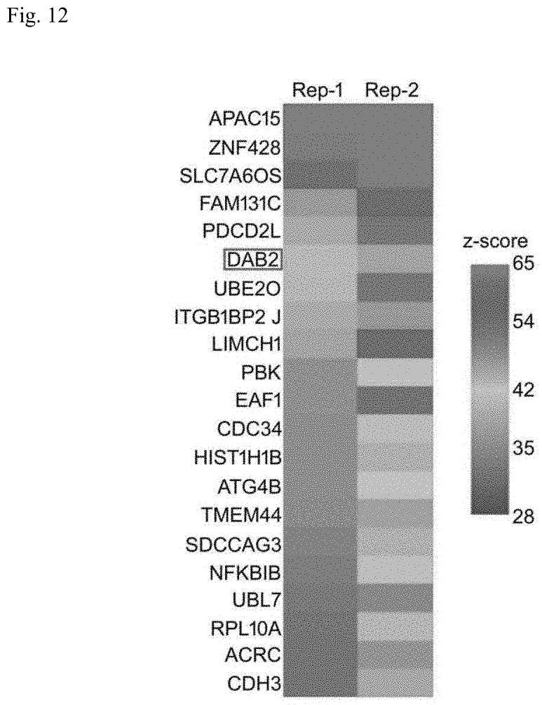

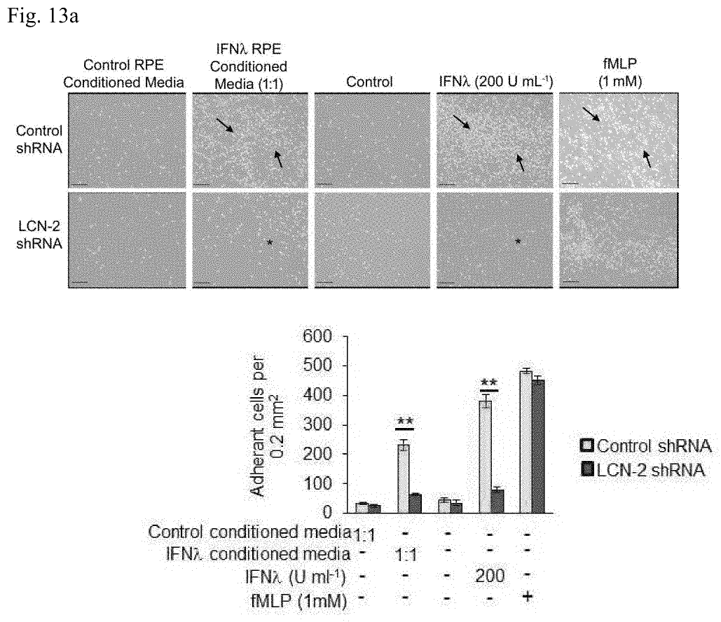

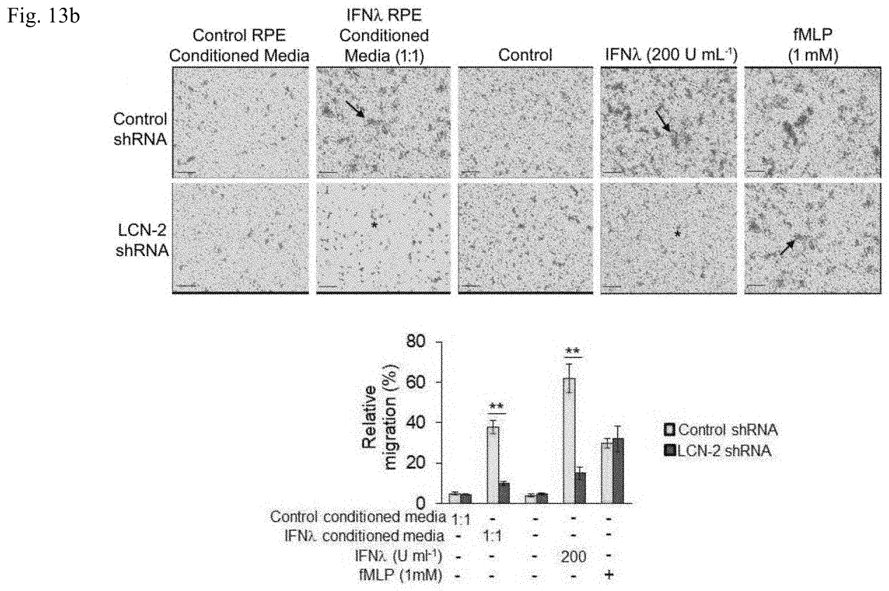

We next performed a proteome high-throughput array and found that LCN-2 interacts with Dab2 (FIG. 12), which is known to regulate cell migration through binding with integrin b1⁴³. Removal of Dab2 inhibits cell migration43. Increased integrin b1 plays a critical role in neutrophil migration^{44, 45} and adhesion^{46, 47}. We observed increased association between LCN-2 and Dab2 in IFNλ, treated neutrophils as compared to controls (FIG. 3C). We hypothesized that the increased association between LCN-2 and Dab2 in IFNλ, treated neutrophils regulates extracellular integrin b1 expression and concomitant neutrophil adhesion and transmigration. To explore this yet unknown role of LCN-2 in regulating the Dab2/integrin b1 axis, we silenced LCN-2 in cultured neutrophils with a specific shRNA to LCN-2 and treated these cells with either recombinant IFNλ, or conditioned medium from IFNλ, overexpressing RPE cells. We found that upon LCN-2 silencing, extracellular integrin b1 expression (FIGS. 3D-3E) and neutrophil adhesion and transmigration (FIGS. 13A-13B), were significantly reduced as compared to cells treated with control shRNA. These results suggest that LCN-2 regulates b1 Integrin-dependent neutrophil adhesion and transmigration.

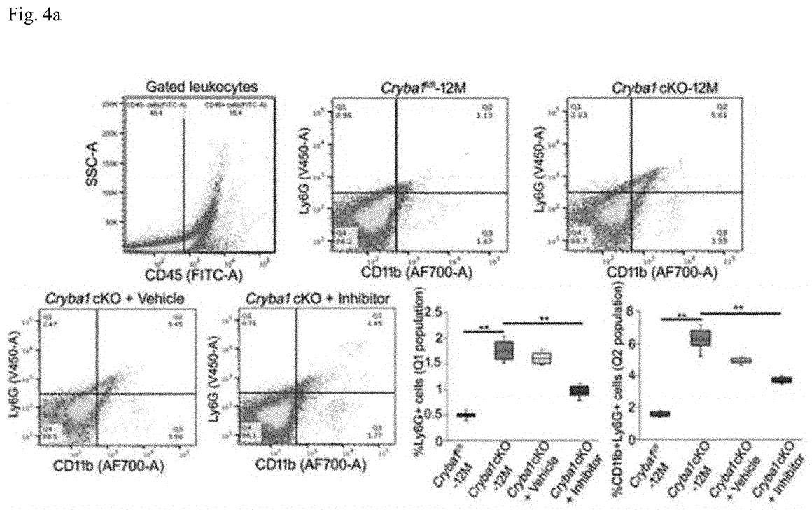

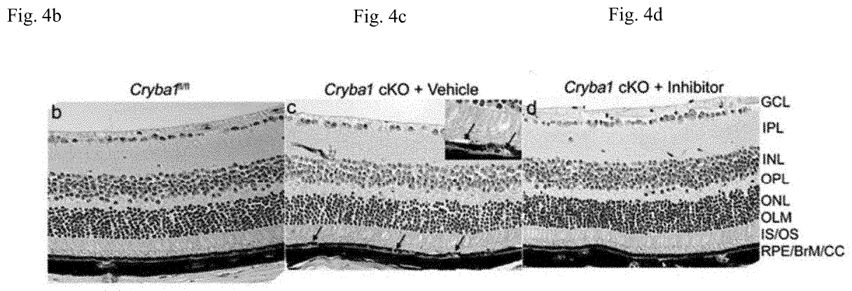

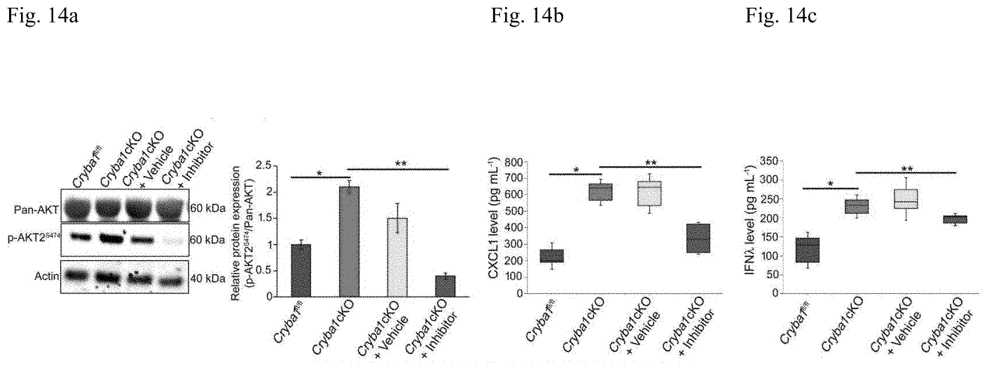

Taken together, our data suggest that neutrophils infiltrating the retina release LCN-2, generating pro-inflammatory conditions⁴² that contribute to elements of AMD pathobiology^{22, 48}. We have previously reported a sharp increase in retinal LCN-2 levels in early AMD patients that persists through the later disease stages²². Since we also demonstrated previously that AKT2 is an upstream regulator of LCN-2²² and herein that LCN2 orchestrates the transmigration of neutrophils into the retina, we next used CCT128930, a potent and selective inhibitor of AKT2⁴⁹ to determine if it could block neutrophil infiltration into the retina in our mouse model. Cryba1 cKO mice exhibit a striking AMD-like phenotype with RPE and photoreceptor degeneration, cardinal changes of early AMD^{50, 51}. The RPE is mildly degenerated at 12 months of age, which progresses to severe RPE and photoreceptor degeneration by 20 months¹⁹⁻²¹. At the early RPE degenerative stage, Cryba1 cKO mice (12 months) injected intravitreally with CCT128930 had significantly fewer neutrophils in the retinas than those given vehicle only (FIG. 4A). Importantly, the drug reverses these early RPE abnormalities (FIGS. 4B-4D). In addition, pAKT2, IFNλ, and CXCL1 levels were reduced by CCT128930 treatment (FIGS. 14A-14C). While antioxidant micronutrients slow intermediate AMD progression^{52, 53} and anti-VEGF injections treat neovascular disease^{54, 55}, no therapy is aimed at the early stages of the disease. We envisage that AKT2 inhibitors are an effective, novel means of preventing or delaying the progression of early AMD.

### Materials and Methods

Antibodies

FITC-tagged CD45 (Cat#553080), APC Cy7-tagged CD45 (Cat#560178), FITC-tagged CD66b (Cat#555724), V450-tagged Ly6G (Cat#560603) and Alexa fluor 700-tagged CD11b (Cat#557960) were purchased from BD Biosciences, USA and PE-tagged IL-28AR antibody (Cat#337804) was purchased from Biolegend, USA. Anti-Neutrophil Elastase (Cat# ab68672), anti-GRO alpha (CXCL1) (Cat# ab86436), anti-STAT1 (phosphor S727) (cat# ab109461), anti-Histone H3 citrunillated (Cat# ab219407) and IL28+IL29 (Cat# ab191426) antibodies were purchased from Abcam, USA. Anti-ICAM-1 (Cat# SC-107). Anti-STAT1 (Cat#9172T), anti-AKT (Cat#4685S), anti-AKT2 (Cat#2964S) and anti-Dab-2 (Cat#12906S) were purchased from Cell Signaling Technologies, USA. Other antibodies used include: Alexa fluor 488-tagged (31 Integrin (Santa Cruz Biotechnology, USA; Cat# sc-374429 AF488), Anti-IL-28A/IFNλ2 (Antibodies online; Cat# ABIN357173), anti-IFNα antibodies (Thermo Fisher, USA; Cat#221001), anti-Myeloperoxidase/MPO (R&D Systems, USA; Cat# AF3667-SP), anti-LCN-2 (EMD Milipore; Cat# AB2267) and anti-Actin (Sigma Aldrich, USA; Cat# A2066).

Animals

βA3/A1-crystallin conditional knockout mice (Cryba1 cKO) were produced using the Cre-LoxP system and the Best1 promoter as described previously¹. Cryba1 floxed mice² were mated with Best1-cre mice that express Cre recombinase specifically in RPE. Offspring that were determined to be cKO+ and Cre+ were subsequently mated together to produce the F2 generation. PCR analysis identified F2 progeny homozygous for the knockout allele. These cKO/cKO mice were subsequently analyzed for presence of Cre. Animals both cKO/cKO and Cre+ were mated to produce the F3 and subsequent generations. The floxed mice were originally bred into the C57BL/6N strain which carries the rd8 mutation, but this retinal degeneration mutation was bred out of the colony before this study was conducted. NOD-SCID mice (NOD.CB17-Prkdescid/J; 4-5 weeks old) were purchased from The Jackson Laboratory, USA. All animal studies were conducted in accordance with the Guide for the Care and Use of Animals (National Academy Press) and were approved by the University of Pittsburgh Animal Care and Use Committee.

Human Eyes

The diagnosis and classification of AMD in human donor eyes was done as previously described³. For immunostaining, human donor eyes were obtained from the National Disease Research Interchange (NDRI; Philadelphia, Pa., USA) within 12-35 h of death. Caucasian donor eyes from 5 subjects with AMD (age range 79-95 years; mean age 85.8 years) and three aged controls (age range 77-89 years; mean age 82.5 years), with no evidence of macular disease were studied. The study adhered to the norms of the Declaration for Helsinki regarding research involving human tissue. The diagnosis of AMD and classification was done as previously described³.

For immunophenotyping and soluble factors quantification experiments from human peripheral blood and aqueous humor, samples were collected from human donors, reporting to Narayana Nethralaya, Bangalore, India. All subjects underwent an ophthalmic exam, including visual acuity testing and retinal examination. AMD patients were diagnosed by fundus imaging, Amsler grid test and optical coherence tomography imaging when deemed necessary. Subjects with co-existing glaucoma or any other degenerative retinal disorders were excluded. The control group consisted of individuals without any history of AMD, diabetes, cardiovascular disorders or retinal diseases. 4-6 ml blood samples were collected from 26 controls and 80 AMD subjects by venipuncture in EDTA tubes. Aqueous humor samples (˜50 μL) were collected from subjects undergoing cataract surgery (n=7 control, n=6 AMD) by anterior chamber paracentesis under sterile conditions. Within this group, early AMD subjects, where surgery is not contra-indicated, were identified by the presence of drusen and RPE abnormalities characterized by pigmentary changes in the retina in accordance with AREDS classification⁴. The demographic characteristics of the cohorts are described in Supplementary Tables 1A-1C. All collected samples were immediately stored in a biorepository for further processing. All patient samples and related clinical information were collected after obtaining approval by the Narayana Nethralaya Institutional Review Board (IRB) and with written, informed consent from patients.

SUPPLEMENTARY TABLE 1A
Periperal blood (Immunophenotyping + Soluble factors)

| | Control (n = 26) | AMD (n = 50) | P value |
|---|---|---|---|
| Age | 64.04 ± 1.91; | 67.82 ± 1.21; | 0.293 |
| (Mean ± SEM; Range) | 43-80 | 49-88 | |
| Years | | | |
| Gender (M/F) | 16/10 | 28/22 | NA |
| Log Mar (BCVA) RE | 0.15 ± 0.08; | 0.34 ± 0.06; | 0.003 |
| | 0-2.09 | 0-1.78 | |
| Log Mar (BCVA) LE | 0.18 ± 0.06; | 0.2 ± 0.03; | 0.299 |
| | 0-1.30 | 0-0.78 | |

SUPPLEMENTARY TABLE 1B
Aqueous humor (Immunophenotyping)

| | Control (n = 7) | AMD (n = 6) | P value |
|---|---|---|---|
| Age | 62.71 ± 2.97; | 63 ± 2.93; | 0.836 |
| (Mean ± SEM; Range) | 53-76 | 55-72 | |
| Years | | | |
| Gender (M/F) | 3/4 | 3/3 | NA |
| Log Mar (BCVA) | 0.51 ± 0.27; | 0.33 ± 0.13; | 0.971 |
| | 0.1-2.09 | 0.03-1 | |

SUPPLEMENTARY TABLE 1C
Aqueous humor (Soluble factors)

| | Control (n = 7) | AMD (n = 6) | P value |
|---|---|---|---|
| Age | 60.43 ± 3.22; | 63 ± 3.58; | 0.462 |
| (Mean ± SEM; Range) | 53-76 | 55-76 | |
| Years | | | |
| Gender (M/F) | 3/4 | 3/3 | NA |
| Log Mar (BCVA) | 0.53 ± 0.27; | 0.23 ± 0.06; | 0.463 |
| | 0.1-2.09 | 0.03-0.48 | |

Immunostaining

Human donor eyes (AMD eyes; n=5, age-matched control; n=3), and freshly enucleated eyes from mice (n=4/group) were fixed in 2% paraformaldehyde (PFA), processed, and sectioned (4 sections per eye) following a previous method⁵. Immunostaining was performed using primary antibodies to Myeloperoxidase/MPO (1:100), Neutrophil Elastase (1:100), ICAM-1 (1:100), VCAM-1 (1:100) or H3 citrunillated histone (1:100), followed by staining with appropriate secondary (1:300) together with DAPI as previously described⁶. Sections were mounted using DAKO Paramount (DAKO Corporation, USA). Images were acquired by a Zeiss LSM 710 confocal workstation.

Soluble Factors Quantification

Peripheral venous blood was obtained by venipuncture (n=80 AMD patients and n=26 control subjects) and aqueous humor (AH) was collected by anterior chamber paracentesis in AMD patients (n=6) and control subjects (n=7). The levels of IFNα, IFNβ, IFNγ, VEGF and CXCL1 were measured in plasma and AH by bead-based multiplex ELISA (BioLegend, Inc, USA) using a flow cytometer (BD FACS Canto II, FACS DIVA software, BD Biosciences, USA). The absolute concentration for each analyte was calculated based on the standard curve.

Immunophenotyping

Cells from peripheral blood (n=80 AMD patients and n=26 control subjects) and aqueous humor (AH) from control subjects (n=7) and AMD patients (n=6), were labeled using fluorochrome conjugated anti-human antibodies specific for leukocytes (CD45), neutrophils (CD66b) and IFNλ, receptor at room temperature for 45 minutes. Red blood cells were lysed in 1×BD lysis buffer for 10 minutes (for peripheral blood samples) and the cells from peripheral blood and AH were washed and resuspended in 1× phosphate buffer saline prior to flow cytometry (BD FACS Canto II, FACS DIVA software, BD Biosciences, USA) based acquisition and analysis. Data were analysed using FCS Express 6 Flow Research Edition software. The leukocyte populations were identified by manual gating using SSC/CD45+ profile. Subsequent gating was done on SSC/CD66b FITC to identify neutrophils. The neutrophil activation status was determined based on CD66b cell surface expression. CD45+CD66b+^{High} cells were considered as activated neutrophils and CD45+CD66b+^{Low} as inactive neutrophils. CD45+CD66b+High/Low IL-28RI+ indicated IFNλ, receptor positive neutrophils. The number of positive cell events for each staining panel was calculated.

RPE Isolation and Culture

Mouse RPE was isolated from control C57BL/6J mice (3 weeks old, n=9; Jackson Laboratories, USA). Eyes were removed and washed twice in 5 ml DMEM containing high glucose and incubated with 5 ml of 2% (wt/vol) Dispase (Sigma Aldrich, USA) in DMEM for 45 min at 37° C. RPE isolation and culture was performed following a previously described method⁷, where two eyes were used per well for appropriate confluency of cells (90%).

IFNλ Overexpression in RPE Cells In Vitro

pLV—C-IL28A-GFPSpark and control vector was purchased from Sino Biological Inc. (Beijing, China, MG51305-ACGLN). Primary mouse RPE cells (in a monolayer; 90% confluent) were transfected with the respective vectors using X-tremeGENE transfection reagent (Roche, Switzerland) following the manufacturer's instructions¹. The transfection efficiency was estimated by evaluating the level of IL-28A/IFNλ, released (into the cell-free supernatant) from overexpression transfected RPE cells, with respect to the control vector transfected cells by ELISA; a minimum of three-fold increase in IL-28A/IFNλ, level was considered appropriate for performing further experiments with the conditioned media.

Isolation and Culture of Neutrophils

Mouse neutrophils were isolated by centrifugation of bone marrow cells, flushed from femurs and tibias and purified over a Percoll discontinuous density gradient in Ca²⁺ and Mg²⁺ free HBSS as previously described⁸. More than 90% of the isolated cells were Ly6G+ neutrophils as determined by flow cytometry (data not shown). Isolated neutrophils were cultured at a density of 5×10⁶ cells/mL, either treated with 100 or 200 U ml⁻¹ of recombinant IFNλ, (R&D Biosystems, USA) or with conditioned media (diluted 1:1 or 1:5 with medium) IFN-λ overexpressing RPE cells, at 37° C. with 5% CO₂ in HBSS containing 20 mM HEPES.

LCN-2 shRNA Transfection

LCN-2 shRNA lentiviral and control shRNA particles were purchased from Santa Cruz Biotechnology, USA (sc-60044-V). Mouse bone marrow derived neutrophils (5×10⁶ cells/mL in HBSS containing 20 mM HEPES) were plated and then transfected with LCN-2 shRNA lentiviral or control shRNA particles for 8 h, according to the manufacturer's protocol, following which, the transfected cells were treated with either 200 U ml⁻¹ of recombinant IFNλ (R&D Biosystems, USA) for 2 h or with IFN-λ overexpressing RPE conditioned media (diluted 1:1 with medium), at 37° C. with 5% CO₂.

Rapid Neutrophil Adhesion Assay

Glass bottom 35 mm plates (Corning, USA) were coated for 16 h at 4° C. with human fibrinogen (Sigma Aldrich, USA) at 20 □g/well in endotoxin-free PBS. Mouse bone marrow derived neutrophils (5×10⁶ cells/mL in HBSS containing 20 mM HEPES medium), previously transfected with control shRNA or NGAL shRNA as explained in the previous section, were treated with fMLP (1 mM), recombinant IFNλ, 200 U ml⁻¹) or□ conditioned media from IFN-λ overexpressing RPE cells. The treated cells were added to coated plates and incubated for 10 min at 37° C., washed with PBS, fixed on ice with 4% paraformaldehyde for 30 mins. The adhering cells were counted in 0.2 mm², using computer-assisted enumeration⁸.

Neutrophil Transmigration Assay

Mouse bone marrow derived neutrophils (5×10⁶ cells/mL in HBSS containing 20 mM HEPES medium) were plated and then transfected with lentiviral LCN-2 shRNA or control shRNA for 8 h (see above). The transfected cells were treated with either 200 U ml⁻¹ of recombinant IFNλ, (R&D Biosystems, USA) or with conditioned media from IFN-λ overexpressing RPE cells (diluted 1:1 with medium), at 37° C. with 5% CO₂. The cells were harvested from the plates, washed in medium, then plated on transwell plates with inserts (Corning, USA) pre-coated with 150 μs/ml of human fibrinogen (Sigma Aldrich, USA). Migrated cells were counted on the bottom of the transwell after staining with Giemsa, by using a computer assisted cell counter⁹.

Estimation of Percentage Neutrophils in Mouse Retina

Mouse retinas were dissected from enucleated eyes and digested with 0.05% collagenase D at 37° C. for 30 min, teased with blunt end forceps and pipetted to release cells, passed through a 70 μm cell strainer, centrifuged at 1,300 g, 4° C. for 20 minutes¹⁰. The entire pellet was used for staining with the FITC-tagged cell surface markers CD45, V450-tagged Ly6G and Alexa fluor 700-tagged CD11b (BD Pharmigen™, USA) at a concentration of 1 μg/ml in PBS containing 1% BSA for 1 h. Cells were washed and analyzed on a flow cytometer (BD Aria III, FACS DIVA software, BD Biosciences, USA and FlowJo software-version 7.6.5), SSC-A/CD45+(FITC) cells were manually gated and among this population of cells; % Ly6G+ and % CD11b+Ly6G+ cells were quantified.

Estimation of Expression of β1-Integrin

Freshly cultured neutrophils were incubated with V450-tagged Ly6G (BD Pharmigen™, USA) and Alexa fluor 488-tagged β1-Integrin (Santa Cruz Biotechnology, USA) antibodies at a concentration of 1 μg/ml in PBS containing 1% BSA for 1 h. Cells were washed with PBS, analyzed by using a flow cytometer (BD Aria III, FACS DIVA software, BD Biosciences, USA and FlowJo software-version 7.6.5). Ly6G+ cells were gated among the total cell population and the cell surface expression of β1-Integrin (FITC fluorescence) was evaluated among the Ly6G+ cells¹¹.

SDS-PAGE and Western Blot Analysis

SDS-PAGE and western blot analyses were performed as previously described¹². The primary antibodies were used at a dilution of 1:1000 whereas, all secondary antibodies were used at a dilution of 1:3000.

Preparation of Recombinant Lipocalin-2 (LCN-2) Protein

Full Length LCN-2 cDNA was synthesized by GeneScipt, USA. It was subcloned in pET28a vector at NdeI and XhoI site. The construct was transformed into E. coli DH5-α cells for amplification and E. coli Rosetta for expression. Single colony was grown overnight as a mother culture. 10% of mother culture was inoculated and grown to 0.8-1.0 OD and induced with 0.5 mM IPTG for 2 h at 37° C. The cells were then pelleted by centrifugation at 6000 rpm for 10 minutes at 4° C. in a microfuge, resuspended in 10% volume of 20 mM Tris pH 8.0, containing 300 mM NaCl and 10% Glycerol. The mixture was sonicated for 30 seconds on and off each for 6 cycles, and then centrifuged at 12000 rpm for 30 minutes at 4° C. The supernatant fraction was passed over a Nickel NTA (BioVision, USA) column as per the manufacturer's protocol. The column was washed twice with 10 times the bed volume with 20 mM Tris pH 8.0, with 300 mM NaCl, 10% Glycerol and 20 mM Imidazole. The protein was eluted with 20 mM Tris pH 8.0, 300 mM NaCl, 10% Glycerol and 300 mM Imidazole with ˜5 times the bed volume in multiple fractions. The protein was dialyzed overnight at 4° C. in 1×PBS and 50% Glycerol and stored at −20° C. in aliquots.

Protein-Protein Interaction

The human proteome microarray 2.0 analysis was performed as a paid service from CDI NextGen Proteomics, MD, USA. Recombinant Lipocalin-2 was analyzed for protein-protein interaction profiling on the HuProt™ v3.1 human proteome array and the sample was probed on array plates at 1 μg/ml, with data analyzed using GenePix software. Hit identification was assessed as the ratio of median value of the foreground to the median of the surrounding background for each protein probe on the microarray, followed by normalization by the median value of all neighboring probes within the 9×9×9 window size and represented as the significance of the probe binding signal difference from random noise (Z-Score). The cut-off Z-score was 6 in this study for the triplicate analysis; only protein interactions with a Z-score above 6 were considered¹².

ELISA

The RPE choroid complexes harvested from freshly enucleated mouse eyes were kept on ice and then homogenized in 300 μL of complete extraction buffer (Abcam, USA) per 5 mg of tissue. The homogenized tissue was allowed to stay in constant agitation for 2 h at 4° C., centrifuged at 13,000 rpm at 4° C. for 20 min. The supernatants were aliquoted and stored at −80° C. and were subsequently used to perform ELISA to evaluate the levels of IFNλ, and CXCL1, as previously described¹³.

RNAseq Analysis

RPE-Choroid from enucleated eyes harvested from 5 and 10 month old Cryba1^{fl/fl} and Cryba1 cKO mice (n=4), respectively, were subjected to total RNA isolation as previously described¹². Approximately 30 ng μV′ total RNA was used to perform RNA-sequencing as a paid service from DNA Link, USA. All sequence reads were mapped to the reference genome (NCBI37/mm9) using the RNA-seq mapping algorithm included in CLC Genomics Workbench. The maximum number of mismatches allowed for the mapping was set at 2. To estimate gene expression levels and analyze for differentially expressed genes among the different groups, RPKM was calculated as previously described¹⁴.

Co-Immunoprecipitation

To evaluate the association between LCN-2 and Dab-2 in different experimental conditions, cultured neutrophils either treated with recombinant IFNλ, (200 U ml⁻¹) or with conditioned media from IFN-λ overexpressing RPE cells (diluted 1:1) were subjected to co-immunoprecipitation (Co-IP) using the Pierce™ Co-Immunoprecipitation Kit (Thermo Fisher, USA, 26149) as previously described¹²,

Intravitreal Injection of AKT2 Inhibitor

Cryba1^{fl/fl} and Cryba1 cKO mice (Male, 12 months old; n=4) were anaesthetized by intraperitoneal injection of 0.15 ml of ketamine (2.5 mg/ml)+ xylazine (0.5 mg/ml) mixture. Topical anesthesia (proparacaine hydrochloride) was applied to the eye and pupils dilated with a drop of topical 2.5% phenylephrine hydrochloride ophthalmic solution. The eye was proptosed by slight depression of the lower lid with blunt curved forceps and washed with sterile saline. For intravitreal injections, a 30-gauge needle was used to make a hole in the eye just posterior to the limbus and then by using a Gastight Syringe (Hamilton robotics, USA) 2 μl inhibitor (500 μM of CCT128930 in 2.5% DMSO in PBS) or vehicle only (2.5% DMSO in PBS) was injected into the vitreous, once every week for three weeks. All instruments were sterilized with a steam autoclave. Bacitracin Ophthalmic ointment was applied postoperatively⁶. Animals were euthanized with CO₂ gas four weeks after the first injection and the retinas were harvested.

Intravitreal Injection of Neutrophils in NOD-SCID Mice and Optical Coherence Tomography (OCT)

NOD-SCID mice (NOD.CB17-Prkdescid/J, Jackson Laboratories, USA, male, 4-5 weeks old) were used for the study. A large sample size, n=10, was taken to nullify any experimental anomaly. Mice were anaesthetized and intravitreal injections performed as described above. HBSS (vehicle control), recombinant LCN-2 (10 pg ml⁻¹) or freshly cultured neutrophils (in HBSS containing 5×10⁴ cells), either untreated or treated with either recombinant IFNλ (200 U ml⁻¹) or IFN-λ overexpressing RPE conditioned media from IFN-λ overexpressing RPE cells, respectively was injected into the vitreous of each eye, once every week for two weeks^{6, 15}.

Three weeks after the first injection, the NOD-SCID mice were anaesthetized by intraperitoneal injection of a ketamine and xylazine mixture and then subjected to Fundus imaging along with Optical Coherence Tomography (OCT) analysis using the Bioptigen Envisu R2210 system. OCT images were analyzed on optical sections (100 sections per retina) from each eye ranging from −2.0 to +2.0 mm with respect to the optic nerve head (ONH) using the FIJI-ImageJ (NIH) plugin provided with the instrument along with Diver 2.4 software (Bioptigen)¹⁶. After the experiment, the animals were euthanized with CO₂ gas and the eyes were harvested for further experiments.

Hematoxylin-Eosin Staining

For hematoxylin and eosin (H&E) staining, eyes from NOD-SCID mice (n=10) were enucleated and fixed initially in 2.5% glutaraldehyde for 72 h, followed by 10% buffered formalin. The eyes were embedded in methyl-methacrylate, sectioned and stained as previously described″.

Statistical Analysis

Statistical analysis was performed with Microsoft Excel and GraphPad Prism 6 software for Windows, by the use of one-way ANOVA. Group means were compared using Tukey's post hoc test, with significance being set at p<0.05. For experiments with human samples, comparisons between control and AMD groups were performed by Mann Whitney test with significance being set at p<0.05, the data distribution was determined by the Shapiro-Wilk normality test. Center lines and edge lines in box plot indicate medians and interquartile range, respectively and whiskers indicate the most extreme data points. The analyses were performed on triplicate technical replicates. Results are presented as mean±standard deviation (SD)³.

### REFERENCES

1. Schmier, J. K., Jones, M. L., Halpern, M. T. The burden of age-related macular degeneration. Pharmacoeconomics. 24, 319-34 (2006).
2. Wong, W. L. et al. Global prevalence of age-related macular degeneration and disease burden projection for 2020 and 2040: a systematic review and meta-analysis. Lancet. Glob Health. 2, e106-116 (2014).
3. Jonas, J. B., Cheung, C. M. G., Panda-Jonas, S. Updates on the Epidemiology of Age-Related Macular Degeneration. Asia Pac J Ophthalmol (Phila). 6, 493-497 (2017).
4. Ambati, J., Atkinson, J. P., Gelfand, B. D. Immunology of age-related macular degeneration. Nat. Rev. Immunol. 13, 438-451 (2013).
5. Guillonneau, X. et al. On phagocytes and macular degeneration. Prog Retin Eye Res. 61, 98-128 (2017).
6. Datta, S. et al. The impact of oxidative stress and inflammation on RPE degeneration in non-neovascular AMD. Prog Retin Eye Res. 60, 201-218 (2017).
7. Copland, D. A., Theodoropoulou, S., Liu, J., Dick, A. D. A Perspective of AMD Through the Eyes of Immunology. Invest Ophthalmol Vis Sci. 59(4), AMD83-AMD92 (2018)
8. Parks, W. C., Wilson, C. L., López-Boado, Y. S. Matrix metalloproteinases as modulators of inflammation and innate immunity. Nat. Rev. Immunol. 4, 617-29 (2004).
9. Kobayashi, S. D., DeLeo, F. R. Role of neutrophils in innate immunity: a systems biology-level approach. Wiley. Interdiscip. Rev. Syst. Biol. Med. 1, 309-333 (2009).
10. Li, P. et al. PAD4 is essential for antibacterial innate immunity mediated by neutrophil extracellular traps. J. Exp. Med. 207, 1853-62 (2010).
11. Massberg, S. et al. Reciprocal coupling of coagulation and innate immunity via neutrophil serine proteases. Nat. Med. 16, 887-96 (2010).
12. Rosales, C., Lowell, C. A., Schnoor, M., Uribe-Querol, E. Neutrophils: Their Role in Innate and Adaptive Immunity 2017. J. Immunol. Res. 2017, U.S. Pat. No. 9,748,345 (2017).
13. Baik, S. H. et al. Migration of neutrophils targeting amyloid plaques in Alzheimer's disease mouse model. Neurobiol. Aging. 35, 1286-92 (2014).
14. Zenaro, E. et al. Neutrophils promote Alzheimer's disease-like pathology and cognitive decline via LFA-1 integrin. Nat. Med. 21, 880-886 (2015).
15. Pietronigro, E. C., Della Bianca, V., Zenaro, E., Constantin, G. NETosis in Alzheimer's Disease. Front. Immunol. 8, 211 (2017).
16. Dong, Y. et al. Neutrophil hyperactivation correlates with Alzheimer's disease progression. Ann. Neurol. 83, 387-405 (2018).
17. Lechner, J. et al. Alterations in Circulating Immune Cells in Neovascular Age-Related Macular Degeneration. Sci. Rep. 5, 16754 (2015).
18. Krogh Nielsen, M., Hector, S. M., Allen, K., Subhi, Y., Sorensen, T. L. Altered activation state of circulating neutrophils in patients with neovascular age-related macular degeneration. Immun. Ageing. 14, 18 (2017).
19. Valapala, M. et al. Lysosomal-mediated waste clearance in retinal pigment epithelial cells is regulated by CRYBA1/βA3/A1-crystallin via V-ATPase-MTORC1 signaling. Autophagy. 10, 480-496 (2014).
20. Shang, P., et al. The amino acid transporter SLC36A4 regulates the amino acid pool in retinal pigmented epithelial cells and mediates the mechanistic target of rapamycin, complex 1 signaling. Aging Cell. 16(2), 349-359 (2017).
21. Ghosh, S. et al. A Role for βA3/A1-Crystallin in Type 2 EMT of RPE Cells Occurring in Dry Age-Related Macular Degeneration. Invest Ophthalmol Vis Sci. 59(4), AMD104-AMD113 (2018).
22. Ghosh, S. et al. Activating the AKT2-nuclear factor-KB-lipocalin-2 axis elicits an inflammatory response in age-related macular degeneration. J Pathol. 241, 583-588 (2017).
23. Choi, E. Y., Santoso, S., Chavakis, T. Mechanisms of neutrophil transendothelial migration. FrontBiosci (Landmark Ed). 14, 1596-605 (2009).
24. Proebstl, D. et al. Pericytes support neutrophil subendothelial cell crawling and breaching of venular walls in vivo. J. Exp. Med. 209, 1219-1234 (2012).
25. Gane, J., Stockley, R. Mechanisms of neutrophil transmigration across the vascular endothelium in COPD. Thorax. 67, 553-61 (2012).
26. Xu N, Hossain M, Liu L. Pharmacological inhibition of p38 mitogen-activated protein kinases affects KC/CXCL1-induced intraluminal crawling, transendothelial migration, and chemotaxis of neutrophils in vivo. Mediators. Inflamm. 2013, 290565 (2013).
27. de Oliveira, S., Rosowski, E. E., Huttenlocher, A. Neutrophil migration in infection and wound repair: going forward in reverse. Nat. Rev. Immunol. 16, 378-91 (2016).
28. Langereis, J. D. Neutrophil integrin affinity regulation in adhesion, migration, and bacterial clearance. Cell. Adh. Migr. 7, 476-81 (2013).
29. Yang, L. et al. ICAM-1 regulates neutrophil adhesion and transcellular migration of TNF-alpha-activated vascular endothelium under flow. Blood. 106, 584-592 (2005).
30. Andrews, R. K., Arthur, J. F., Gardiner, E. E. Neutrophil extracellular traps (NETs) and the role of platelets in infection. Thromb. Haemost. 112, 659-65 (2014).
31. Gestermann, N. et al. Netting Neutrophils Activate Autoreactive B Cells in Lupus. J.

Immunol. 200, 3364-3371 (2018).
32. Cervantes-Luevano, K. E. et al. Neutrophils drive type I interferon production and autoantibodies in patients with Wiskott-Aldrich syndrome. J. Allergy. Clin. Immunol. (2018).
33. McDonald, B. et al. Platelets and neutrophil extracellular traps collaborate to promote intravascular coagulation during sepsis in mice. Blood. 129, 1357-1367 (2017).
34. Yazdani, H. O. et al. IL-33 exacerbates liver sterile inflammation by amplifying neutrophil extracellular trap formation. Hepatol. (2017).
35. Paunel-Görgülü, A. et al. cfDNA correlates with endothelial damage after cardiac surgery with prolonged cardiopulmonary bypass and amplifies NETosis in an intracellular TLR9-independent manner. Sci. Rep. 7, 17421 (2017).
36. van der Windt, D. J. et al. Neutrophil extracellular traps promote inflammation and development of hepatocellular carcinoma in nonalcoholic steatohepatitis. Hepatology. (2018).
37. Odobasic, D., Kitching, A. R., Semple, T. J., Holdsworth, S. R. Endogenous myeloperoxidase promotes neutrophil-mediated renal injury but attenuates T cell immunity inducing crescentic glomerulonephritis. J. Am. Soc. Nephrol. 18, 760-70 (2007).
38. Fujie, K. et al. Release of neutrophil elastase and its role in tissue injury in acute inflammation: effect of the elastase inhibitor, FR134043. Eur. J. Pharmacol. 374, 117-25 (1999).
39. Sahoo, M., Del Barrio, L., Miller, M. A., Re, F. Neutrophil elastase causes tissue damage that decreases host tolerance to lung infection with burkholderia species. PLoS Pathog. 10, e1004327 (2014).
40. Yang, R., Zou, X., Tenhunen, J., Tønnessen, T. I. HMGB1 and Extracellular Histones Significantly Contribute to Systemic Inflammation and Multiple Organ Failure in Acute Liver Failure. Mediators Inflamm. 2017, U.S. Pat. No. 5,928,078 (2017).
41. Valapala, M. et al. Impaired endolysosomal function disrupts Notch signalling in optic nerve astrocytes. Nat. Commun. 4,1629 (2013).
42. Valapala, M. et al. Increased Lipocalin-2 in the retinal pigment epithelium of Cryba1 cKO mice is associated with a chronic inflammatory response. Aging cell. 13, 1091-4 (2014).
43. Teckchandani, A. et al. Quantitative proteomics identifies a Dab2/integrin module regulating cell migration. J. Cell. Biol. 186, 99-111 (2009).
44. van den Berg, J. M. et al. Beta1 integrin activation on human neutrophils promotes beta2 integrin-mediated adhesion to fibronectin. Eur. J. Immunol. 31(1), 276-284 (2001).
45. Silveira, A. A. A. et al. TNF induces neutrophil adhesion via formin-dependent cytoskeletal reorganization and activation of β-integrin function. J. Leukoc. Biol. 103, 87-98 (2018).
46. Sarangi, P. P., Hyun, Y. M., Lerman, Y. V., Pietropaoli, A. P., Kim, M. Role of (31 integrin in tissue homing of neutrophils during sepsis. Shock. 38, 281-287 (2012).
47. Hanlon, S. D., Smith, C. W., Sauter, M. N., Burns, A. R. Integrin-dependent neutrophil migration in the injured mouse cornea. Exp. Eye. Res. 120, 61-70 (2014).
48. Parmar, T. et al. Lipocalin 2 Plays an Important Role in Regulating Inflammation in Retinal Degeneration. J Immunol. 2018 May 1; 200(9):3128-3141.
49. Yap, T. A., et al. Preclinical pharmacology, antitumor activity, and development of pharmacodynamic markers for the novel, potent AKT inhibitor CCT128930. Mol Cancer Ther. 10(2), 360-371 (2011).
50. Bonilha, V. L. Age and disease-related structural changes in the retinal pigment epithelium. Clin. Ophthalmol. 2, 413-24 (2008).
51. Wang, J. et al. ATAC-Seq analysis reveals a widespread decrease of chromatin accessibility in age-related macular degeneration. Nat. Commun. 9, 1364 (2018).
52. Age-Related Eye Disease Study Research Group. A randomized, placebo-controlled, clinical trial of high-dose supplementation with vitamins C and E, beta carotene, and zinc for age-related macular degeneration and vision loss: AREDS report no. 8. Arch. Ophthalmol. 126, 1251 (2008).
53. Chew, E. Y. et al. Long-term effects of vitamins C and E, β-carotene, and zinc on age-related macular degeneration: AREDS report no. 35. Ophthalmology. 120, 1604-11.e4 (2013).
54. Nguyen, C. L., Oh, L. J., Wong, E., Wei, J., Chilov, M. Anti-vascular endothelial growth factor for neovascular age-related macular degeneration: a meta-analysis of randomized controlled trials. BMC. Ophthalmol. 18, 130 (2018).
55. Adrean, S. D., Chaili, S., Ramkumar, H., Pirouz, A., Grant, S. Consistent Long-Term Therapy of Neovascular Age-Related Macular Degeneration Managed by 50 or More Anti-VEGF Injections Using a Treat-Extend-Stop Protocol. Ophthalmology. 125, 1047-1053 (2018).

1-4. (canceled)

5. A method for treating ocular vascular disease comprising administering an effective amount of an Akt inhibitor to a subject in need thereof.

6. The method of claim 5, wherein the ocular vascular disease is selected from the group consisting of age-related macular degeneration, wet age-related macular degeneration, retinitis pigmentosa, diabetic retinopathy and geographic atrophy.

7. The method of claim 5, wherein the Akt inhibitor is an Akt2 inhibitor.

8. The method of claim 7, wherein the Akt inhibitor is a small molecule.

9. The method of claim 8, wherein the Akt inhibitor is orally administered.

10. The method of claim 6, wherein the Akt inhibitor is an Akt2 inhibitor.

11. The method of claim 10, wherein the Akt inhibitor is a small molecule.

12. The method of claim 11, wherein the Akt inhibitor is orally administered.

13. The method of claim 5, wherein the Akt inhibitor is an mRNA interfering RNA molecule, double-stranded RNA, short interfering RNA, short hairpin RNA, an antisense oligonucleotide, or an antibody antagonist of Akt protein.

14. The method of claim 5, wherein the Akt inhibitor is an Akt2 selective and/or Akt2 specific inhibitor.

15. The method of claim 5, wherein the Akt inhibitor is CCT128930.

16. The method of claim 5, wherein the Akt inhibitor is administered in combination with one or more additional therapeutic agents.

17. The method of claim 6, wherein the Akt inhibitor is an mRNA interfering RNA molecule, double-stranded RNA, short interfering RNA, short hairpin RNA, an antisense oligonucleotide, or an antibody antagonist of Akt protein.

18. The method of claim 6, wherein the Akt inhibitor is an Akt2 selective and/or Akt2 specific inhibitor.

19. The method of claim 6, wherein the Akt inhibitor is CCT128930.

20. The method of claim 6, wherein the Akt inhibitor is administered in combination with one or more additional therapeutic agents.
